(19) United States
(12) Patent Application Publication
(10) Pub. No.: US 2026/0108445 A1
(43) Pub. Date: Apr. 23, 2026

(54) CREAMY SILICONE AND SULFATE-FREE CONDITIONING SHAMPOO

(71) Applicant: L'OREAL, Paris (FR)

(72) Inventors: Jianxin FENG, Clark, NJ (US); Manon CHAUMONTET, Jersey City, NJ (US); Nicole MASIUK, Clark, NJ (US)

(73) Assignee: L'OREAL, Paris (FR)

(21) Appl. No.: 18/919,957

(22) Filed: Oct. 18, 2024

(51) Int. Cl. A61K 8/46; A61K 8/44; A61Q 5/02

(52) U.S. Cl. CPC A61K 8/466; A61K 8/44; A61Q 5/02; A61K 2800/432; A61K 2800/436

(57) ABSTRACT

A cleansing and conditioning composition, free from silicones and free from sulfate-based surfactants, is disclosed comprising: (a) a plurality of non-sulfate anionic surfactants, the plurality of non-sulfate anionic surfactants comprising one or more isethionate surfactants, one or more amino acid surfactants, and one or more alkoxylated monoacid cosurfactants; (b) one or more amphoteric surfactants; (c) optionally, one or more nonionic surfactants; and (d) water. The cleansing composition is particularly effective as a conditioning shampoo, useful for shampooing and conditioning the hair.

### FIELD OF THE DISCLOSURE

The present disclosure pertains to creamy cleansing compositions with conditioning properties that are free or essentially free from sulfate-based surfactants. The cleansing compositions are useful for cleansing the hair and body, and especially effective for shampooing and simultaneously conditioning the hair.

### BACKGROUND

Cleansing compositions, including shampoos and body washes, are crucial for removing dirt, oil, and grease from skin and hair. The effectiveness of these products hinges on the inclusion of surfactants, which are essential functional ingredients. Surfactants interact with water to enhance its ability to wet surfaces more thoroughly, facilitating the removal of oil and grease. This interaction allows the surfactant-water mixture to effectively surround and lift away dirt, which is then rinsed off. Agitation, such as rubbing hands together during washing or massaging shampoo into the hair, further aids in dislodging and removing contaminants.

Typically, cleansing compositions contain a variety of surfactants. Anionic surfactants are frequently used due to their superior foaming properties, which contribute to the product's cleansing effectiveness by lifting and suspending dirt and oil. Conversely, nonionic surfactants, while also effective in cleansing and solubilizing, generally produce less foam and are known for being milder and less irritating. However, nonionic surfactants can result in a thinner, runnier product, which may not be desirable when a higher viscosity is preferred.

Recent trends indicate a growing consumer preference for formulations that avoid silicones and sulfate-based surfactants. Silicones, used to provide a smooth and shiny appearance in hair care products, can accumulate over time, leaving hair feeling heavy or greasy. This has led to an increased demand for silicone-free formulations. Additionally, sulfate-based surfactants, which are known for their strong foaming and cleansing capabilities, are being avoided due to their potential to cause dryness or irritation. Sulfates can strip away natural oils, leading to dryness and discomfort, which many consumers wish to avoid.

The development of cleansing compositions must address these evolving consumer preferences while balancing various performance characteristics. The ideal formulation should ensure effective cleansing, desirable foaming, appropriate viscosity, and mildness to skin and hair, all without using silicones or sulfate-based surfactants. Achieving this balance is complex, as enhancing one property may impact others. For instance, increasing viscosity could affect foamability or rinseability. Therefore, the formulation process requires careful consideration to meet consumer demands while managing these performance trade-offs.

The formulation of environmentally-friendly cosmetic products, which are designed and developed considering environmental issues, is becoming a major goal in an effort to meet global challenges. It is therefore essential to propose more sustainable compositions and ingredients to address these environmental concerns. In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

### SUMMARY OF THE DISCLOSURE

The present invention pertains to novel cleansing compositions that effectively cleanse and condition hair without the need for sulfate-based surfactants. The cleansing compositions may be characterized as a “shampoo,” “a conditioning shampoo,” or an “all-in-one shampoo.” Nonetheless, the cleansing compositions are not limited to shampoos as they are useful for cleansing other surfaces, for example, the body and skin.

Traditional shampoos often rely on sulfate-based surfactants like sodium lauryl sulfate (SLS) and sodium laureth sulfate (SLES) for their powerful cleansing and foaming properties. However, these surfactants can cause scalp irritation and dryness by stripping away essential natural oils, which are vital for maintaining healthy hair. For individuals with sensitive skin or scalp conditions such as eczema or psoriasis, the sulfate-free formulations described herein offer a gentler alternative, preserving the scalp's natural moisture balance.

The cleansing composition typically includes:
(a) about 2 to about 25 wt. % of a plurality of non-sulfate anionic surfactants, the plurality of non-sulfate anionic surfactants comprising:
   (i) about 1 to about 10 wt. % of one or more isethionate surfactants;
   (ii) about 0.1 to about 10 wt. % of one or more amino acid surfactants;
   (iii) about 0.1 to about 5 wt. % of one or more alkoxylated monoacid cosurfactants;
(b) about 2 to about 10 wt. % of one or more amphoteric surfactants;
(c) optionally, one or more nonionic surfactants;
(d) about 60 to about 90 wt. % of water;
   all percentages by weight based on a total weight of the cleansing composition.

The cleansing composition is preferably essentially free or free from sulfate-based anionic surfactants.

Nonlimiting examples of isethionate surfactants include sodium cocoyl isethionate, sodium lauroyl isethionate, sodium myristoyl isethionate, sodium capryloyl isethionate, sodium stearoyl isethionate, sodium oleoyl isethionate, sodium palmitoyl isethionate, sodium lauroyl methyl isethionate, sodium cocoyl methyl isethionate, sodium decanoyl isethionate, sodium lauroyl sarcosinate, sodium cetyl isethionate, sodium isethionate, sodium cocoate isethionate, sodium hexanoyl isethionate, sodium tetradecanoyl isethionate, sodium nonanoyl isethionate, sodium oleoyl methyl isethionate, sodium isethionate laurate, sodium isethionate stearate, sodium dodecanoyl isethionate, sodium palmitoleoyl isethionate, sodium isethionate caprate, sodium isethionate linoleate, sodium lauryl isethionate, sodium caproyl isethionate, and sodium isethionate behenate.

Nonlimiting examples of amino acid surfactants include sarcosinate surfactants, taurate surfactants, glycinate surfactants, and glutamate surfactants. In a preferred embodiment, at least one of the one or more amino acid surfactants is a sarcosinate surfactant. Nonlimiting examples of sarcosinate surfactants include sodium lauroyl sarcosinate, sodium cocoyl sarcosinate, sodium myristoyl sarcosinate, sodium palmitoyl sarcosinate, sodium stearoyl sarcosinate, sodium oleoyl sarcosinate, sodium linoleoyl sarcosinate, sodium linolenoyl sarcosinate, sodium capryloyl sarcosinate, sodium caproyl sarcosinate, sodium lauric acid sarcosinate, sodium lauroyl methyl sarcosinate, sodium cocoyl methyl sarcosinate, sodium myristoyl methyl sarcosinate, sodium palmitoyl methyl sarcosinate, sodium stearoyl methyl sarcosinate, sodium oleoyl methyl sarcosinate, sodium linoleoyl methyl sarcosinate, sodium linolenoyl methyl sarcosinate, sodium capryloyl methyl sarcosinate, sodium caproyl methyl sarcosinate, sodium lauryl sarcosinate, sodium cocoyl sarcosinate, sodium myristyl sarcosinate, sodium palmityl sarcosinate, sodium stearyl sarcosinate, sodium oleyl sarcosinate, sodium linoleyl sarcosinate, sodium linolenyl sarcosinate, sodium caprylyl sarcosinate, and sodium caprolyl sarcosinate.

Nonlimiting examples of alkoxylated monoacid cosurfactants include trideceth-7 carboxylic acid, sodium laureth-13 carboxylate, sodium laureth-4 carboxylate, laureth-11 carboxylic acid, laureth-5 carboxylic acid, sodium laureth-5 carboxylate, ammonium laureth-6 carboxylate, ammonium laureth-8 carboxylate, capryleth-4 carboxylic acid, capryleth-6 carboxylic acid, capryleth-9 carboxylic acid, ceteareth-25 carboxylic acid, cetyl c12-15 pareth-8 carboxylate, cetyl C12-15 pareth-9 carboxylate, cetyl ppg-2 isodeceth-7 carboxylate, coceth-7 carboxylic acid, C9-11 pareth-6 carboxylic acid, C9-11 pareth-8 carboxylic acid, C11-15 pareth-7 carboxylic acid, C12-13 pareth-5 carboxylic acid, C12-13 pareth-7 carboxylic acid, C12-13 pareth-8 carboxylic acid, C12-13 pareth-12 carboxylic acid, C12-15 pareth-7 carboxylic acid, C12-15 pareth-8 carboxylic acid, C12-15 pareth-12 carboxylic acid, C14-15 pareth-8 carboxylic acid, deceth-7 carboxylic acid, ethylhexeth-3 carboxylic acid, hexeth-4 carboxylic acid, isopropyl C12-15-pareth-9 carboxylate, isopropyl ppg-2 isodeceth-7 carboxylate, isosteareth-6 carboxylic acid, isosteareth-11 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, magnesium laureth-11 carboxylate, mea-laureth-6 carboxylate, mea ppg-6 laureth-7 carboxylate, mea-ppg-8-stearath-7 carboxylate, myreth-3 carboxylic acid, myreth-5 carboxylic acid, oleth-3 carboxylic acid, oleth-6 carboxylic acid, oleth-10 carboxylic acid, peg-2 stearamides carboxylic acid, peg-9 stearamide carboxylic acid, potassium laureth-3 carboxylate, potassium laureth-4 carboxylate, potassium laureth-5 carboxylate, potassium laureth-6 carboxylate, potassium laureth-10 carboxylate, potassium trideceth-3 carboxylate, potassium trideceth-4 carboxylate, potassium trideceth-7 carboxylate, potassium trideceth-15 carboxylate, potassium trideceth-19 carboxylate, ppg-3-deceth-2 carboxylic acid, propyl c12-15 pareth-8 carboxylate, sodium capryleth-2 carboxylate, sodium capryleth-9 carboxylate, sodium ceteareth-13 carboxylate, sodium ceteth-13 carboxylate, sodium C9-11 pareth-6 carboxylate, sodium C11-15 pareth-7 carboxylate, sodium C12-13 pareth-5 carboxylate, sodium C12-13 pareth-8 carboxylate, sodium C12-13 pareth-12 carboxylate, sodium C12-15 pareth-6 carboxylate, sodium C12-15 pareth-7 carboxylate, sodium C12-15 pareth-8 carboxylate, sodium C12-15 pareth-12 carboxylate, sodium C14-15 pareth-8 carboxylate, sodium C12-14 sec-pareth-8 carboxylate, sodium deceth-2 carboxylate, sodium hexeth-4 carboxylate, sodium isosteareth-6 carboxylate, sodium isosteareth-11 carboxylate, sodium laureth-3 carboxylate, sodium laureth-4 carboxylate, sodium laureth-5 carboxylate, sodium laureth-6 carboxylate, sodium laureth-8 carboxylate, sodium laureth-11 carboxylate, sodium laureth-12 carboxylate, sodium laureth-13 carboxylate, sodium laureth-14 carboxylate, sodium laureth-16 carboxylate, sodium laureth-17 carboxylate, sodium lauryl glucose carboxylate, sodium lauryl glycol carboxylate, sodium peg-6 cocamide carboxylate, sodium peg-8 cocamide carboxylate, sodium peg-3 lauramide carboxylate, sodium peg-4 lauramide carboxylate, sodium peg-7 olive oil carboxylate, sodium peg-8 palm glycerides carboxylate, sodium trideceth-3 carboxylate, sodium trideceth-4 carboxylate, sodium trideceth-6 carboxylate, sodium trideceth-7 carboxylate, sodium trideceth-8 carboxylate, sodium trideceth-12 carboxylate, sodium trideceth-15 carboxylate, sodium trideceth-19 carboxylate, sodium undeceth-5 carboxylate, trideceth-3 carboxylic acid, trideceth-4 carboxylic acid, trideceth-7 carboxylic acid, trideceth-8 carboxylic acid, trideceth-15 carboxylic acid, trideceth-19 carboxylic acid and undeceth-5 carboxylic acid.

Amphoteric surfactants include, but are not limited to, betaine surfactants, sultaine surfactants, amphopropionate surfactants, and amphoacetate surfactants. In various embodiments, the cleansing composition preferably includes at least one betaine surfactant. Nonlimiting examples of betaine surfactants include coco betaine, cocamidopropyl betaine, lauryl betaine, laurylhydroxy sulfobetaine, lauryldimethyl betaine, behenyl betaine, capryl/capramidopropyl betaine, stearyl betaine, lauramidopropyl betaine, myristamidopropyl betaine, palmitamidopropyl betaine, stearamidopropyl betaine, oleamidopropyl betaine, capramidopropyl betaine, caprylamidopropyl betaine, behenamidopropyl betaine, lauramidopropyl betaine, cocamidopropyl hydroxysultaine, and cocamidopropyl dimethylamine betaine.

Nonionic surfactants can be alkoxylated or non-alkoxylated. Nonlimiting examples of nonionic surfactants include alkanolamides; alkyl polyglucosides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄) acylaminopropylmorpholine oxides; and combinations thereof.

The cleansing composition optionally includes one or more cationic conditioning polymers. Nonlimiting examples of cationic conditioning polymers include cationic polysaccharide derivatives (cationic polysaccharide compounds), cationic gum derivatives (cationic gum compounds), polyquaterniums, polymer derivatives of diallyldimethyl ammonium chloride, polymer derivatives of methacrylamidopropyltrimethylammonium chloride, cationic cellulose derivatives (cationic cellulose compounds), quaternized hydroxyethyl cellulose, cationic starch derivatives (cationic starch compounds), cationic guar gum derivatives (cationic guar compounds), copolymers of acrylamide and dimethyldiallyammonium chloride, and a mixture thereof.

In various embodiments, the cleansing composition preferably includes one or more cationic guar compounds, one or more polyquaterniums, or combinations thereof. Nonlimiting examples of cationic guar compounds include guar hydroxypropyltrimonium chloride, guar hydroxypropyltrimethylammoniumchloride (GPTC), hydroxypropyl guar hydroxypropyltrimonium chloride, guar hydroxypropyltrimethylammonium chloride, and hydroxyethylguar hydroxypropyltrimonium chloride.

In various embodiments, the cleansing composition preferably includes one or more polyquaterniums. Nonlimiting examples of polyquaterniums include polyquaternium-4, polyquaternium-7, polyquaternium-10, polyquaternium-11, polyquaternium-22, polyquaternium-37, polyquaternium-44, or combinations thereof. In a preferred embodiment, the additional cationic polymer is a polyquaternium that is cellulose based. Nonlimiting examples include Polyquaternium-4, polyquaternium-7, polyquaternium-10, polyquaternium-11, polyquaternium-22, polyquaternium-28, polyquaternium-33, and polyquaternium-37.

The cleansing composition optionally includes one or more pearlizing agents. Nonlimiting examples of pearlizing agents include glycol distearate, PEG-150 distearate, sorbitan stearate, mica, titanium dioxide, alumina, and combinations thereof.

The cleansing composition optionally includes one or more direct dyes. Nonlimiting examples of direct dyes generally include azo direct dyes; anthraquinone and anthraquinone derivatives; (poly) methine dyes such as cyanins, hemicyanins and styryls; carbonyl dyes; azine dyes; nitro (hetero) aryl dyes; tri (hetero) arylmethane dyes; porphyrin dyes; phthalocyanin dyes, and natural direct dyes, alone or as mixtures. The direct dyes may be cationic or anionic.

The cleansing composition does not require certain polyols, such as glycerin and glycols, for example, polyols having from 2 to 10 carbon atoms. Nonetheless, in various embodiments, it can be useful to includes one or more polyols, such as glycerin and glycols. Nonlimiting examples include glycerol, ethylene glycol, propylene glycol (1,2-propanediol), 1,3-propanediol, butylene glycol (1,3-butanediol), 1,2-butanediol, 2,3-butanediol, pentylene glycol, 1,2-pentanediol, 2,3-pentanediol, 3,4-pentanediol, hexylene glycol, 1,2-hexanediol, 1,3-hexanediol, and 2,3-hexanediol.

The cleansing composition optionally includes one or more thickening polymers. Nonlimiting examples of thickening polymers include polyacrylate polymers, polyacrylamide polymers, polysaccharides, gums (e.g., guar gums), carbomers, and a combination thereof.

The cleansing composition optionally includes salicylic acid. Salicylic acid helps to remove scalp buildup, treat dandruff and seborrheic dermatitis, control oily scalp, and promote healthy scalp and hair.

The pH of the cleansing composition is typically from about 4 to less than 7. Preferably, however, the pH is from about 4.5 to about 6.5, more preferably the pH is from about 5.5 to about 6.5.

The cleansing compositions are particularly useful for cleansing and conditioning hair. The compositions exhibit good cleansing ability, lather, foaming and foam stability, and conditioning properties. Additionally, the cleansing compositions provide a variety of desirable benefits to the hair, for example, smoothness, detangling, and shine. Accordingly, the cleansing compositions may be used in methods for cleansing hair, methods of conditioning hair, and methods for imparting smoothness, detangling, and/or shine to hair.

### DETAILED DESCRIPTION OF THE DISCLOSURE

The cleansing compositions of the present disclosure provide effective hair cleansing without the use of sulfate-based surfactants and deliver excellent conditioning without relying on silicones. Due to their cleansing and conditioning properties, they may be referred to “cleansing and conditioning compositions.” The compositions are particularly useful for cleansing and conditioning hair, including human hair of the head. Thus, they may be characterized as a “shampoo” or a “conditioning shampoo.” Nonetheless, they are effective for cleansing other surfaces and are not required to be a shampoo.

The cleansing composition typically comprises:
(a) about 2 to about 20 wt. % of a plurality of non-sulfate anionic surfactants, the plurality of non-sulfate anionic surfactants comprising:
   (i) about 1 to about 10 wt. % of one or more isethionate surfactants;
   (ii) about 0.1 to about 10 wt. % of one or more amino acid surfactants; and
   (iii) about 0.1 to about 5 wt. % of one or more alkoxylated monoacid cosurfactants;
(b) about 2 to about 10 wt. % of one or more amphoteric surfactants;
(c) less than 10 wt. % of one or more nonionic surfactants;
(d) about 60 to about 90 wt. % of water;
   all percentages by weight based on a total weight of the cleansing composition.

The cleansing composition is preferably free or essentially free from sulfate-based anionic surfactants. In addition, the cleansing composition may be free or essentially free from ethanol and/or propanol, or the cleansing composition may be free or essentially free from mono-alcohols with 2 to 7 carbon atoms. Nonlimiting examples include ethanol, propanol, isopropanol, butanol, isobutanol, pentanol, hexanol, and heptanol. The cleansing composition may also be free or essentially free from silicones.

### (a) Non-Sulfate Anionic Surfactants

Useful non-sulfate anionic surfactants include, but are not limited to, isethionate surfactants, amino acid surfactants (taurate surfactants, glycinate surfactants, glutamate surfactants, and sarcosinate surfactants), sulfonate surfactants, sulfosuccinate surfactants, sulfoacetate surfactants, alkoxylated monoacid cosurfactants, salts thereof, and a mixture thereof. More detailed descriptions and nonlimiting examples of useful non-sulfate anionic surfactants are provided below.

### (a) (i) Isethionate Surfactants

Nonlimiting examples of useful isethionate surfactants include those of formula (I) and (II):

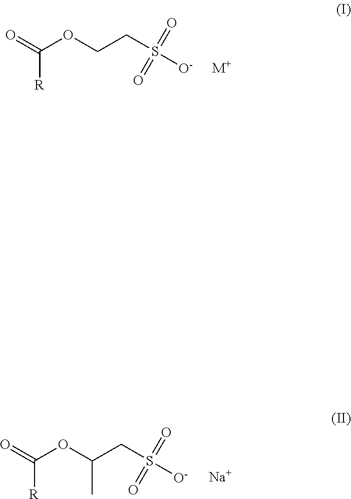

wherein R, R¹, R² and R³ are each independently selected from H or an alkyl chain having 1-24 carbon atoms, said chain being saturated or unsaturated, linear, or branched, and X is COO⁻ or SO₃⁻. Sodium is shown as the cation in formula (VI) but the cation for both formula (V) and formula (VI) may be an alkali metal ion such as sodium or potassium, ammonium ions, or alkanolammonium ions such as monoethanolammonium or triethanolammonium ions.

Nonlimiting examples of isethionate surfactants include sodium cocoyl isethionate, sodium lauroyl isethionate, sodium myristoyl isethionate, sodium capryloyl isethionate, sodium stearoyl isethionate, sodium oleoyl isethionate, sodium palmitoyl isethionate, sodium lauroyl methyl isethionate, sodium cocoyl methyl isethionate, sodium decanoyl isethionate, sodium lauroyl sarcosinate, sodium cetyl isethionate, sodium isethionate, sodium cocoate isethionate, sodium hexanoyl isethionate, sodium tetradecanoyl isethionate, sodium nonanoyl isethionate, sodium oleoyl methyl isethionate, sodium isethionate laurate, sodium isethionate stearate, sodium dodecanoyl isethionate, sodium palmitoleoyl isethionate, sodium isethionate caprate, sodium isethionate linoleate, sodium lauryl isethionate, sodium caproyl isethionate, and sodium isethionate behenate. In a preferred embodiment, one or more isethionate surfactants are selected from sodium isethionate, sodium cocoyl isethionate, sodium lauroyl methyl isethionate, and sodium cocoyl methyl isethionate. In some instances, sodium cocoyl isethionate and/or sodium cocoyl methyl isethionate are particularly preferred isethionate surfactants that may be included in the cleansing compositions.

The total amount of the one or more isethionate surfactants in the cleansing composition will vary but is typically from about 1 to about 10 wt. %, based on the total weight of the cleansing composition. In further embodiments, the cleansing composition includes about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, about 3 to about 5 wt. %, or about 4 wt. % of one or more isethionate surfactants, based on the total weight of the cleansing composition. Preferably, the cleansing composition includes from about 1 to about 10 wt. %, more preferably about 2 to about 8 wt. %, more preferably about 2 to about 6 wt. %, and even more preferably about 3 to about 6 wt. % of one or more isethionate surfactants, based on the total weight of the cleansing composition.

(a)(ii) Amino Acid Surfactants

Amino acid surfactants are a particularly useful type of non-sulfate anionic surfactant for use in the instant cleansing compositions. Useful amino acid surfactants that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the amino acid surfactants can be sodium or potassium. Alternatively, the cation can be an organic salt such as triethanolamine (TEA) or a metal salt. Nonlimiting examples of useful amino acid surfactants include those of formula (III)

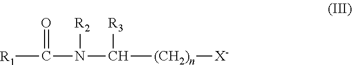

wherein R, R¹, R² and R³ are each independently selected from H or an alkyl chain having 1-24 carbon atoms, said chain being saturated or unsaturated, linear, or branched, and X is COO⁻ or SO₃⁻. In some cases, one or more taurate surfactants and/or sarcosinate surfactants are preferred.

The total amount of the one or more amino acid surfactants in the cleansing composition will vary but is typically from about 1 to about 10 wt. %, based on the total weight of the cleansing composition. In further embodiments, the cleansing composition includes about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. % of one or more amino acid surfactants, based on the total weight of the cleansing composition. Preferably, the cleansing composition includes from about 1 to about 10 wt. %, more preferably about 2 to about 8 wt. %, more preferably about 2 to about 6 wt. %, and even more preferably about 3 to about 5 wt. % of one or more amino acid surfactants, based on the total weight of the cleansing composition.

Sarcosinate Surfactants: Sarcosinate surfactants are a particularly useful type of amino acid surfactant for use in the cleansing compositions.

Nonlimiting examples of sarcosinate surfactants include sodium lauroyl sarcosinate, sodium cocoyl sarcosinate, sodium myristoyl sarcosinate, sodium palmitoyl sarcosinate, sodium stearoyl sarcosinate, sodium oleoyl sarcosinate, sodium linoleoyl sarcosinate, sodium linolenoyl sarcosinate, sodium capryloyl sarcosinate, sodium caproyl sarcosinate, sodium lauric acid sarcosinate, sodium lauroyl methyl sarcosinate, sodium cocoyl methyl sarcosinate, sodium myristoyl methyl sarcosinate, sodium palmitoyl methyl sarcosinate, sodium stearoyl methyl sarcosinate, sodium oleoyl methyl sarcosinate, sodium linoleoyl methyl sarcosinate, sodium linolenoyl methyl sarcosinate, sodium capryloyl methyl sarcosinate, sodium caproyl methyl sarcosinate, sodium lauryl sarcosinate, sodium cocoyl sarcosinate, sodium myristyl sarcosinate, sodium palmityl sarcosinate, sodium stearyl sarcosinate, sodium oleyl sarcosinate, sodium linoleyl sarcosinate, sodium linolenyl sarcosinate, sodium caprylyl sarcosinate, and sodium caprolyl sarcosinate. In a preferred embodiment, the one or more sarcosinate surfactants are selected from potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate. In some instances, sodium lauroyl sarcosinate is

The total amount of the one or more sarcosinate surfactants in the cleansing composition will vary but is typically from about 1 to about 10 wt. %, based on the total weight of the cleansing composition. In further embodiments, the cleansing composition includes about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. % of one or more sarcosinate surfactants, based on the total weight of the cleansing composition. Preferably, the cleansing composition includes from about 1 to about 10 wt. %, more preferably about 2 to about 8 wt. %, more preferably about 2 to about 6 wt. %, and even more preferably about 3 to about 5 wt. % of one or more sarcosinate surfactants, based on the total weight of the cleansing composition.

Taurate Surfactants: Nonlimiting examples of taurate surfactants include those of formula (IV):

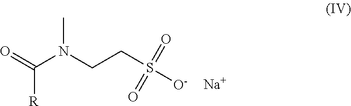

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear, or branched, and X is COO⁻ or SO₃⁻.

The total amount of the one or more taurate surfactants in the cleansing composition, if present, will vary but is typically from about 1 to about 10 wt. %, based on the total weight of the cleansing composition. In further embodiments, the cleansing composition includes about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. % of one or more taurate surfactants, based on the total weight of the cleansing composition. Preferably, the cleansing composition includes from about 1 to about 10 wt. %, more preferably about 2 to about 8 wt. %, more preferably about 2 to about 6 wt. %, and even more preferably about 3 to about 5 wt. % of one or more taurate surfactants, based on the total weight of the cleansing composition.

Glycinate Surfactants: Nonlimiting examples of useful glycinate surfactants include those of formula (V):

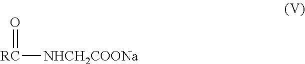

wherein R is an alkyl chain of 8 to 16 carbon atoms. Sodium is shown as the cation in the above formula (V) but the cation may be an alkali metal ion such as sodium or potassium, ammonium ions, or alkanolammonium ions such as monoethanolammonium or triethanolammonium ions. Nonlimiting examples of glycinate surfactants include sodium cocoyl glycinate, sodium lauroyl glycinate, sodium myristoyl glycinate, potassium lauroyl glycinate, and potassium cocoyl glycinate, and in particular sodium cocoyl glycinate.

The total amount of the one or more glycinate surfactants in the cleansing composition, if present, will vary but is typically from about 1 to about 10 wt. %, based on the total weight of the cleansing composition. In further embodiments, the cleansing composition includes about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. % of one or more glycinate surfactants, based on the total weight of the cleansing composition. Preferably, the cleansing composition includes from about 1 to about 10 wt. %, more preferably about 2 to about 8 wt. %, more preferably about 2 to about 6 wt. %, and even more preferably about 3 to about 5 wt. % of one or more glycinate surfactants, based on the total weight of the cleansing composition.

Glutamate Surfactants: Nonlimiting examples of useful glutamate surfactants include those of formula (VI):

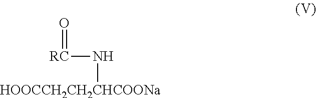

wherein R is an alkyl chain of 8 to 16 carbon atoms. Sodium is shown as the cation in the above formula (VI) but the cation may be an alkali metal ion such as sodium or potassium, ammonium ions, or alkanolammonium ions such as monoethanolammonium or triethanolammonium ions. Nonlimiting examples of gluatamte surfactants include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

The total amount of the one or more glutamate surfactants in the cleansing composition, if present, will vary but is typically from about 1 to about 10 wt. %, based on the total weight of the cleansing composition. In further embodiments, the cleansing composition includes about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. % of one or more glutamate surfactants, based on the total weight of the cleansing composition. Preferably, the cleansing composition includes from about 1 to about 10 wt. %, more preferably about 2 to about 8 wt. %, more preferably about 2 to about 6 wt. %, and even more preferably about 3 to about 5 wt. % of one or more glutamate surfactants, based on the total weight of the cleansing composition.

(a)(iii) Alkoxylated Monoacid Surfactants

For purposes of the instant disclosure, the term “alkoxylated monoacid cosurfactants” includes alkoxylated carboxylate cosurfactants. Alkoxylated carboxylate surfactants are surfactants that contain a carboxylate group (the deprotonated form of a carboxylic acid) and have been modified with alkylene oxide units, such as ethylene oxide. The surfactant may exist in the cleansing composition as a carboxylic acid, a carboxylate (the deprotonated form of the carboxylic acid), or in an equilibrium between the protonated and deprotonated form. Thus, the term “alkoxylated monoacid cosurfactant” encompasses both the protonated (carboxylic acid) and deprotonated form (carboxylate) form. For example, reference to “laureth-5-carboxylic acid” includes “laureth-5 carboxylate” and vice versa.

Nonlimiting examples of alkoxylated monoacid cosurfactants include compounds corresponding to formula (VII):

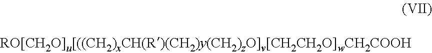

wherein: R is a hydrocarbon radical containing from about 6 to about 40 carbon atoms;
u, v, and w, independently of one another, represent numbers of from 0 to 60;
x, y, and z, independently of one another, represent numbers of from 0 to 13;
R′ represents hydrogen, alkyl, and
the sum of x+y+z>0.

Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated, or unsaturated, aliphatic, or aromatic, substituted, or unsubstituted. Typically, R is a linear or branched, acyclic C6-40 alkyl or alkenyl group or a C1-40 alkyl phenyl group, more typically a C8-22 alkyl or alkenyl group or a C4-18 alkyl phenyl group, and even more typically a C12-18 alkyl group or alkenyl group or a C6-16 alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Nonlimiting examples of alkoxylated monoacid cosurfactants include trideceth-7 carboxylic acid, sodium laureth-13 carboxylate, sodium laureth-4 carboxylate, laureth-11 carboxylic acid, laureth-5 carboxylic acid, sodium laureth-5 carboxylate, ammonium laureth-6 carboxylate, ammonium laureth-8 carboxylate, capryleth-4 carboxylic acid, capryleth-6 carboxylic acid, capryleth-9 carboxylic acid, ceteareth-25 carboxylic acid, cetyl c12-15 pareth-8 carboxylate, cetyl C12-15 pareth-9 carboxylate, cetyl ppg-2 isodeceth-7 carboxylate, coceth-7 carboxylic acid, C9-11 pareth-6 carboxylic acid, C9-11 pareth-8 carboxylic acid, C11-15 pareth-7 carboxylic acid, C12-13 pareth-5 carboxylic acid, C12-13 pareth-7 carboxylic acid, C12-13 pareth-8 carboxylic acid, C12-13 pareth-12 carboxylic acid, C12-15 pareth-7 carboxylic acid, C12-15 pareth-8 carboxylic acid, C12-15 pareth-12 carboxylic acid, C14-15 pareth-8 carboxylic acid, deceth-7 carboxylic acid, ethylhexeth-3 carboxylic acid, hexeth-4 carboxylic acid, isopropyl C12-15-pareth-9 carboxylate, isopropyl ppg-2 isodeceth-7 carboxylate, isosteareth-6 carboxylic acid, isosteareth-11 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, magnesium laureth-11 carboxylate, mea-laureth-6 carboxylate, mea ppg-6 laureth-7 carboxylate, mea-ppg-8-stearath-7 carboxylate, myreth-3 carboxylic acid, myreth-5 carboxylic acid, oleth-3 carboxylic acid, oleth-6 carboxylic acid, oleth-10 carboxylic acid, peg-2 stearamides carboxylic acid, peg-9 stearamide carboxylic acid, potassium laureth-3 carboxylate, potassium laureth-4 carboxylate, potassium laureth-5 carboxylate, potassium laureth-6 carboxylate, potassium laureth-10 carboxylate, potassium trideceth-3 carboxylate, potassium trideceth-4 carboxylate, potassium trideceth-7 carboxylate, potassium trideceth-15 carboxylate, potassium trideceth-19 carboxylate, ppg-3-deceth-2 carboxylic acid, propyl c12-15 pareth-8 carboxylate, sodium capryleth-2 carboxylate, sodium capryleth-9 carboxylate, sodium ceteareth-13 carboxylate, sodium ceteth-13 carboxylate, sodium C9-11 pareth-6 carboxylate, sodium C11-15 pareth-7 carboxylate, sodium C12-13 pareth-5 carboxylate, sodium C12-13 pareth-8 carboxylate, sodium C12-13 pareth-12 carboxylate, sodium C12-15 pareth-6 carboxylate, sodium C12-15 pareth-7 carboxylate, sodium C12-15 pareth-8 carboxylate, sodium C12-15 pareth-12 carboxylate, sodium C14-15 pareth-8 carboxylate, sodium C12-14 sec-pareth-8 carboxylate, sodium deceth-2 carboxylate, sodium hexeth-4 carboxylate, sodium isosteareth-6 carboxylate, sodium isosteareth-11 carboxylate, sodium laureth-3 carboxylate, sodium laureth-4 carboxylate, sodium laureth-5 carboxylate, sodium laureth-6 carboxylate, sodium laureth-8 carboxylate, sodium laureth-11 carboxylate, sodium laureth-12 carboxylate, sodium laureth-13 carboxylate, sodium laureth-14 carboxylate, sodium laureth-16 carboxylate, sodium laureth-17 carboxylate, sodium lauryl glucose carboxylate, sodium lauryl glycol carboxylate, sodium peg-6 cocamide carboxylate, sodium peg-8 cocamide carboxylate, sodium peg-3 lauramide carboxylate, sodium peg-4 lauramide carboxylate, sodium peg-7 olive oil carboxylate, sodium peg-8 palm glycerides carboxylate, sodium trideceth-3 carboxylate, sodium trideceth-4 carboxylate, sodium trideceth-6 carboxylate, sodium trideceth-7 carboxylate, sodium trideceth-8 carboxylate, sodium trideceth-12 carboxylate, sodium trideceth-15 carboxylate, sodium trideceth-19 carboxylate, sodium undeceth-5 carboxylate, trideceth-3 carboxylic acid, trideceth-4 carboxylic acid, trideceth-7 carboxylic acid, trideceth-8 carboxylic acid, trideceth-15 carboxylic acid, trideceth-19 carboxylic acid and undeceth-5 carboxylic acid.

In a preferred embodiment, one or more alkoxylated monoacid cosurfactants are selected from butoxynol-5 carboxylic acid, butoxynol-19 carboxylic acid, capryleth-4 carboxylic acid, capryleth-6 carboxylic acid, capryleth-9 carboxylic acid, ceteareth-25 carboxylic acid, coceth-7 carboxylic acid, C9-11 pareth-6 carboxylic acid, C11-15 pareth-7 carboxylic acid, C12-13 pareth-5 carboxylic acid, C12-13 pareth-8 carboxylic acid, C12-13 pareth-12 carboxylic acid, C12-15 pareth-7 carboxylic acid, C12-15 pareth-8 carboxylic acid, C14-15 pareth-8 carboxylic acid, deceth-7 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, PPG-6-laureth-6 carboxylic acid, PPG-8-steareth-7 carboxylic acid, myreth-3 carboxylic acid, myreth-5 carboxylic acid, nonoxynol-5 carboxylic acid, nonoxynol-8 carboxylic acid, nonoxynol-10 carboxylic acid, octeth-3 carboxylic acid, octoxynol-20 carboxylic acid, oleth-3 carboxylic acid, oleth-6 carboxylic acid, oleth-10 carboxylic acid, PPG-3-deceth-2 carboxylic acid, capryleth-2 carboxylic acid, ceteth-13 carboxylic acid, deceth-2 carboxylic acid, hexeth-4 carboxylic acid, isosteareth-6 carboxylic acid, isosteareth-11 carboxylic acid, trudeceth-3 carboxylic acid, trideceth-6 carboxylic acid, trideceth-8 carboxylic acid, trideceth-12 carboxylic acid, trideceth-4 carboxylic acid, trideceth-7 carboxylic acid, trideceth-15 carboxylic acid, trideceth-19 carboxylic acid, undeceth-5 carboxylic acid, and mixtures thereof.

In some cases, preferred alkoxylated monoacid cosurfactants in laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, oleth-3 carboxylic acid, oleth-6 carboxylic acid, oleth-10 carboxylic acid, trideceth-6 carboxylic acid, trideceth-8 carboxylic acid, trideceth-12 carboxylic acid, trideceth-4 carboxylic acid, trideceth-7 carboxylic acid, trideceth-15 carboxylic acid, trideceth-19 carboxylic acid, and combinations thereof. Particularly preferable alkoxylated monoacid cosurfactants include oleth-10 carboxylic acid, laureth-5 carboxylic acid, laureth-11 carboxylic acid, and combinations thereof.

The total amount of the one or more alkoxylated monoacid cosurfactants in the cleansing composition, if present, will vary but is typically from about 0.1 to about 10 wt. %, based on the total weight of the cleansing composition. In further embodiments, the cleansing composition includes about 0.1 to about 8 wt. %, about 0.1 to about 6 wt. %, about 0.1 to about 4 wt. %, about 0.1 to about 2 wt. %, about 0.2 to about 10 wt. %, about 0.2 to about 6 wt. %, about 0.2 to about 4 wt. %, about 0.5 to about 2 wt. %, about 0.5 to about 10 wt. %, about 0.5 to about 8 wt. %, about 0.5 to about 6 wt. %, about 0.5 to about 4 wt. %, about 0.5 to about 3 wt. %, or about 0.5 to about 2 wt. % of the one or more alkoxylated monoacid cosurfactants, based on the total weight of the cleansing composition. Preferably, the cleansing composition includes from about 0.1 to about 10 wt. %, more preferably about 0.2 to about 8 wt. %, more preferably about 0.2 to about 5 wt. %, and even more preferably about 0.5 to about 3 wt. % of one or more alkoxylated monoacid cosurfactants, based on the total weight of the cleansing composition.

Combined Amounts of (a)(ii) and (a)(iii)

The combined amount of the one or more amino acid surfactants (a) (ii) and the one or more alkoxylated monoacid surfactant (a) (iii) will vary. Nonetheless, the combined amount of (a) (ii) and (a) (iii) is typically from about 0.2 to about 15 wt. %, based on the total weight of the cleansing composition. In further embodiments, the cleansing composition comprises a combined amount of (a) (ii) and (a) (iii) of about 0.2 to about 10 wt. %, about 0.2 to about 8 wt. %, about 0.2 to about 5 wt. %, about 0.2 to less than 5 wt. %, about 0.2 to about 4 wt. %, about 0.2 to about 3 wt. %, about 0.5 to about 15 wt. %, about 0.5 to about 10 wt. %, about 0.5 to about 8 wt. %, about 0.5 to about 5 wt. %, about 0.5 to less than 5 wt. %, about 0.5 to about 4 wt. %, about 0.5 to about 3 wt. %, about 1 to about 15 wt. %, about 1 to about 10 wt. %, about 1 to about 8 wt. %, about 1 to about 5 wt. %, about 1 to less than 5 wt. %, about 1 to about 4 wt. %, about 1 to about 3 wt. %, about 1.5 to about 15 wt. %, about 1.5 to about 10 wt. %, about 1.5 to about 8 wt. %, about 1.5 to about 5 wt. %, about 1.5 to less than 5 wt. %, about 1.5 to about 4 wt. %, or about 1.5 to about 3 wt. % of a combination of (a) (ii) and (a) (iii), based on the total weight of the cleansing composition. Preferably, the cleansing composition comprises about 0.2 to about 10 wt. %, more preferably about 0.5 to less than 5 wt. %, even more preferably about 0.5 to about 4 wt. %, and even more preferably about 1 to about 3 wt. % of a combination of (a) (ii) and (a) (iii), based on the total weight of the cleansing composition.

(a)(iv) Miscellaneous Non-Sulfate Surfactants

The cleansing compositions optionally include one or more miscellaneous anionic surfactants. A miscellaneous anionic surfactant is a non-sulfate surfactant other than the one or more isethionate surfactants and the one or more amino acid surfactants. Nonlimiting examples of miscellaneous anionic surfactants include sulfonate surfactants, sulfosuccinate surfactants, and sulfoacetate surfactants.

The total amount of the one or more one or more miscellaneous anionic surfactants in the cleansing composition, if present, will vary but is typically from about 1 to about 10 wt. %, based on the total weight of the cleansing composition. In further embodiments, the cleansing composition includes about 1 to about 8 wt. %, about 1 to about 6 wt. %, about 1 to about 5 wt. %, about 2 to about 10 wt. %, about 2 to about 8 wt. %, about 2 to about 6 wt. %, about 2 to about 5 wt. %, about 3 to about 10 wt. %, about 3 to about 8 wt. %, about 3 to about 6 wt. %, or about 3 to about 5 wt. % of one or more miscellaneous anionic surfactants, based on the total weight of the cleansing composition. Preferably, the cleansing composition includes from about 1 to about 10 wt. %, more preferably about 2 to about 8 wt. %, more preferably about 2 to about 6 wt. %, and even more preferably about 3 to about 5 wt. % of one or more miscellaneous anionic surfactants, based on the total weight of the cleansing composition.

### (1) Sulfonate Surfactants

Useful sulfonate surfactants include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, sulfonate surfactants of formula (VIII) are particularly useful.

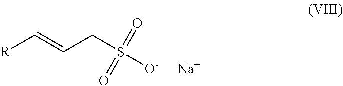

R is selected from H or alkyl chain that has 1-24 carbon atoms, preferably 6-24 carbon atoms, more preferably, 8 to 20 carbon atoms, said chain being saturated or unsaturated, linear, or branched. Sodium is shown as the cation in the above formula but the cation may be an alkali metal ion such as sodium or potassium, ammonium ions, or alkanolammonium ions such as monoethanolammonium or triethanolammonium ions. In some instances, the sulfonate surfactants are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates are particularly preferred. A nonlimiting but particularly useful example of a C₁₀-C₂₄ olefin sulfonate that can be used in the instant compositions is sodium C14-16 olefin sulfonate.

The total amount of the one or more sulfonate surfactants in the cleansing composition, if present, will vary but is typically from about 0.1 to about 10 wt. %, based on the total weight of the cleansing composition. In further embodiments, the cleansing composition includes about 0.1 to about 8 wt. %, about 0.1 to about 6 wt. %, about 0.1 to about 4 wt. %, about 0.5 to about 10 wt. %, about 0.5 to about 8 wt. %, about 0.5 to about 5 wt. %, about 0.5 to about 4 wt. %, about 1 to about 10 wt. %, about 1 to about 8 wt. %, about 1 to about 5 wt. %, about 1 to about 4 wt. %, about 2 to about 10 wt. %, about 2 to about 8 wt. %, or about 2 to about 5 wt. % of one or more sulfonate surfactants, based on the total weight of the cleansing composition. Preferably, the cleansing composition includes from about 0.1 to about 10 wt. %, more preferably about 0.2 to about 8 wt. %, more preferably about 0.5 to about 6 wt. %, and even more preferably about 1 to about 5 wt. % of one or more sulfonate surfactants, based on the total weight of the cleansing composition.

### (2) Sulfosuccinate Surfactants

Nonlimiting examples of sulfosuccinate surfactants include those of formula (IX):

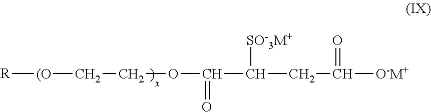

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Nonlimiting examples of sulfosuccinate surfactants salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

The total amount of the one or more sulfosuccinate surfactants in the cleansing composition, if present, will vary but is typically from about 0.1 to about 10 wt. %, based on the total weight of the cleansing composition. In further embodiments, the cleansing composition includes about 0.1 to about 8 wt. %, about 0.1 to about 6 wt. %, about 0.1 to about 4 wt. %, about 0.5 to about 10 wt. %, about 0.5 to about 8 wt. %, about 0.5 to about 5 wt. %, about 0.5 to about 4 wt. %, about 1 to about 10 wt. %, about 1 to about 8 wt. %, about 1 to about 5 wt. %, about 1 to about 4 wt. %, about 2 to about 10 wt. %, about 2 to about 8 wt. %, or about 2 to about 5 wt. % of one or more sulfosuccinate surfactants, based on the total weight of the cleansing composition. Preferably, the cleansing composition includes from about 0.1 to about 10 wt. %, more preferably about 0.2 to about 8 wt. %, more preferably about 0.5 to about 6 wt. %, and even more preferably about 1 to about 5 wt. % of one or more sulfosuccinate surfactants, based on the total weight of the cleansing composition.

### (3) Sulfoacetate Surfactants

Nonlimiting examples of sulfoacetate surfactants include alkyl sulfoacetates such as C4-C18 fatty alcohol sulfoacetates and/or salts thereof. More specific but nonlimiting examples of sulfoacetate surfactants include sodium lauroyl sulfoacetate, sodium coco-sulfate, sodium caproyl sulfoacetate, sodium myristoyl sulfoacetate, potassium lauroyl sulfoacetate, calcium lauroyl sulfoacetate, ammonium lauroyl sulfoacetate, sodium stearoyl sulfoacetate, sodium oleoyl sulfoacetate, sodium palmitoyl sulfoacetate, sodium lauryl sulfoacetate, sodium capryloyl sulfoacetate, sodium heptanoic sulfoacetate, and sodium decanoyl sulfoacetate are a comprehensive list of anionic sulfoacetate surfactants. In various embodiments, a particularly preferred sulfoacetate surfactant is sodium lauryl sulfoacetate. Useful cations for the salts include alkali metal ions such as sodium or potassium, ammonium ions, or alkanolammonium ions such as monoethanolammonium or triethanolammonium ions.

The total amount of the one or more sulfoacetate surfactants in the cleansing composition, if present, will vary but is typically from about 0.1 to about 10 wt. %, based on the total weight of the cleansing composition. In further embodiments, the cleansing composition includes about 0.1 to about 8 wt. %, about 0.1 to about 6 wt. %, about 0.1 to about 4 wt. %, about 0.5 to about 10 wt. %, about 0.5 to about 8 wt. %, about 0.5 to about 5 wt. %, about 0.5 to about 4 wt. %, about 1 to about 10 wt. %, about 1 to about 8 wt. %, about 1 to about 5 wt. %, about 1 to about 4 wt. %, about 2 to about 10 wt. %, about 2 to about 8 wt. %, or about 2 to about 5 wt. % of one or more sulfoacetate surfactants, based on the total weight of the cleansing composition. Preferably, the cleansing composition includes from about 0.1 to about 10 wt. %, more preferably about 0.2 to about 8 wt. %, more preferably about 0.5 to about 6 wt. %, and even more preferably about 1 to about 5 wt. % of one or more sulfoacetate surfactants, based on the total weight of the cleansing composition.

### (b) Amphoteric Surfactants

Amphoteric surfactants are a class of surfactants that possess both positive and negative charges within their molecular structure, allowing them to exhibit dual behavior depending on the pH of the surrounding environment. These surfactants can behave like cationic or anionic surfactants based on the pH, making them highly versatile. The term “amphoteric surfactant” includes “zwitterionic surfactants,” which are a subset of amphoteric surfactants characterized by having both positive and negative charges on different parts of the same molecule, but with no net charge overall.

Nonlimiting examples of amphoteric surfactants include betaine surfactants, amphopropionate surfactants, sultaine surfactants, and amphoacetate surfactants.

The total amount of the one or more amphoteric surfactants in the cleansing compositions will vary but is typically from about 0.1 to about 15 wt. %, based on the total weight of the cleansing composition. In further embodiments, the cleansing composition includes about 0.1 to about 12 wt. %, about 0.1 to about 10 wt. %, from about 0.1 to about 8 wt. %, about 0.1 to about 5 wt. %, about 0.1 to about 3 wt. %, about 0.5 to about 15 wt. %, about 0.5 to about 12 wt. %, about 0.5 to about 10 wt. %, from about 0.5 to about 8 wt. %, about 0.5 to about 5 wt. %, about 0.5 to about 3 wt. %, 1 to about 12 wt. %, about 1 to about 10 wt. %, from about 1 to about 8 wt. %, about 1 to about 5 wt. %, about 1 to about 3 wt. %, about 2 to about 15 wt. %, about 2 to about 12 wt. %, about 2 to about 10 wt. %, about 2 to about 8 wt. %, about 2 to about 6 wt. %, about 4 to about 15 wt. %, about 4 to about 12 wt. %, about 4 to about 10 wt. %, or about 4 to about 8 wt. % of one or more amphoteric surfactants based on the total weight of the cleansing composition. Preferably, the cleansing composition includes from about 0.1 to about 15 wt. %, more preferably from about 0.5 to about 12 wt. %, and more preferably from about 1 to about 10 wt. %, and even more preferably from about 3 to about 8 wt. % of one or more amphoteric surfactants.

The weight ratio of the total amount of the (a) plurality of non-sulfate anionic surfactants to the total amount of the (b) one or more amphoteric surfactant will vary but is typically from about 3:1 to about 1:3 ((a):(b)). In further embodiments, the weight ratio of (a) to (b) is from about 2:1 to about 1:2, or about 1.5:1 to about 1:1.5. In a preferred embodiment, the total amount of the (b) one or more amphoteric surfactants is greater than the total amount of the (a) the one or more non-sulfate anionic surfactants.

(b)(i) Betaine Surfactants

Nonlimiting examples of betaine surfactants include those of formulae (Xa), (Xb), (Xc), and (xd):

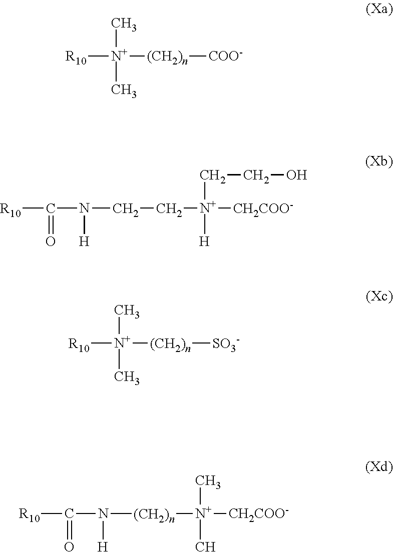

wherein Rio is an alkyl group having 8-18 carbon atoms; and n is an integer from 1 to 3.

Nonlimiting examples of betaine surfactants include cocamidopropyl betaine, lauramidopropyl betaine, capramidopropyl betaine, myristamidopropyl betaine, oleamidopropyl betaine, stearamidopropyl betaine, decyl betaine, dodecyl betaine, coco betaine, hydroxyethyl betaine, lauryl betaine, coco betaine, capryloyl glycine betaine, glyceryl betaine, behenamidopropyl betaine, palmitamidopropyl betaine, isostearamidopropyl betaine, cocamidopropyl hydroxysultaine, undecyl betaine, laurylamidopropyl betaine, caprylamidopropyl betaine, cocoamidopropyl hydroxysultaine, myristylamidopropyl betaine, stearyl betaine, hydroxypropyl betaine, alkyl betaine, and behenyl betaine. Preferably, the one or more betaine surfactants include at least one of cocamidopropyl betaine, lauryl betaine, coco betaine, and myristamidopropyl betaine.

The total amount of the one or more betaine surfactants in the cleansing compositions will vary but is typically from about 0.1 to about 20 wt. %, based on the total weight of the cleansing composition. In further embodiments, the cleansing composition includes about 0.1 to about 15 wt. %, about 0.1 to about 12 wt. %, about 0.1 to about 10 wt. %, about 0.5 to about 20 wt. %, about 0.5 to about 15 wt. %, about 0.5 to about 12 wt. %, about 0.5 to about 10 wt. %, about 1 to about 20 wt. %, about 1 to about 15 wt. %, about 1 to about 12 wt. %, about 1 to about 10 wt. %, about 2 to about 20 wt. %, about 2 to about 15 wt. %, about 2 to about 12 wt. %, about 2 to about 10 wt. %, about 5 to about 20 wt. %, about 4 to about 15 wt. %, about 4 to about 12 wt. %, about 4 to about 10 wt. %, about 5 to about 20 wt. %, about 5 to about 15 wt. %, about 5 to about 12 wt. %, about 5 to about 10 wt. %, about 8 to about 20 wt. %, about 8 to about 15 wt. %, about or about 8 to about 12 wt. % of one or more betaine surfactants based on the total weight of the cleansing composition. Preferably, the cleansing composition includes from about 1 to about 18 wt. %, more preferably from about 2 to about 15 wt. %, and more preferably from about 5 to about 12 wt. % of one or more betaine surfactants, based on the total weight of the cleansing composition.

(b)(ii) Amphopropionate Surfactants

Amphopropionate surfactants are a category of amphoteric surfactants that contain a propionate group in their molecular structure. The presence of the propionate group contributes to their mildness and compatibility with other ingredients, making them suitable for use in the cleansing compositions of the present disclosure. Nonlimiting examples of amphopropionate surfactants include cocoamphopropionate, cornamphopropionate, caprylamphopropionate, cornamphopropionate, caproamphopropionate, oleoamphopropionate, isostearoamphopropionate, stearoamphopropionate, lauroamphopropionate, decylamphopropionate, lauramidopropyl amphopropionate, myristamidopropyl amphopropionate, cetylamphopropionate, and behenamphopropionate. In some instances, sodium cocoamphopropionate is a particularly useful.

(b)(iii) Sultaine Surfactants

Sultaine surfactants are a class of amphoteric surfactants characterized by the presence of a sulfonate group (typically a sulfoalkyl group) in their molecular structure. The term “sultaine” comes from the combination of “sulfonate” and “quaternary ammonium,” reflecting their chemical nature. Nonlimiting examples of sultaine surfactants include sultaines of formula (XI)

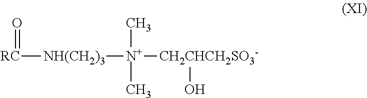

wherein R is an alkyl group having 8-18 carbon atoms. More specific examples include, but are not limited to cocamidopropyl hydroxysultaine, lauryl hydroxysultaine, and a combination thereof. In addition to cocamidopropyl hydroxysultaine and lauryl hydroxysultaine, other sultaine surfactants include capramidopropyl hydroxysultaine, myristamidopropyl hydroxysultaine, oleamidopropyl hydroxysultaine, stearamidopropyl hydroxysultaine, and decyl hydroxysultaine.
   (b)(iv) Amphoacetates and Amphodiacetate Surfactants

Amphoacetates are a class of amphoteric surfactants characterized by the presence of acetate groups in their molecular structure. The acetate group in these surfactants contributes to their ability to interact with both anionic and nonionic surfactants, providing a balanced performance in personal care products. Amphodiacetates are similar to amphoacetates, but contain both acetate and carboxylate groups in their structure. Nonlimiting examples include the compounds of Formula (XII) and (XIII):

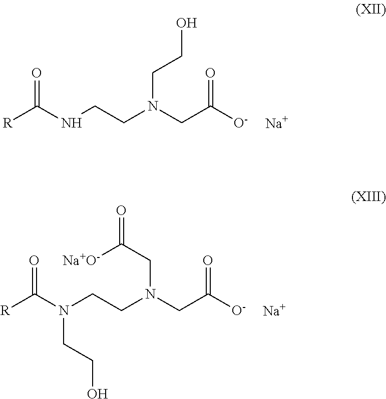

wherein R is an alkyl group having 8-18 carbon atoms. Sodium is shown as the cation in the formulae above, but the cation may be an alkali metal ion such as sodium or potassium, ammonium ions, or alkanolammonium ions such as monoethanolammonium or triethanolammonium ions.

Nonlimiting examples of amphoacetate surfactants include sodium cocoamphoacetate, sodium lauramphoacetate, potassium cocoamphoacetate, sodium oleamphoacetate, sodium capramphoacetate, and sodium stearamphoacetate. Nonlimiting examples of amphodiacetate surfactants include sodium cocoamphodiacetate, sodium lauramphodiacetate, potassium cocoamphodiacetate, sodium myristamphodiacetate, sodium isostearamphodiacetate, and potassium lauramphodiacetate.

### (c) Nonionic Surfactants

The cleansing compositions may optionally include one or more nonionic surfactants. Nonlimiting examples of nonionic surfactants include: alkanolamides; alkyl polyglucosides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄) acylaminopropylmorpholine oxides; and combinations thereof.

The total amount of the one or more nonionic surfactants in the cleansing composition, if present, will vary. Nonetheless, the total amount of the one or more nonionic surfactants is typically less than 10 wt. %, based on the total weight of the cleansing composition. In further embodiments, the cleansing composition includes less than 8 wt. %, less than 6 wt. %, less than 5 wt. %, less than 4 wt. %, less than 3 wt. %, less than 2 wt. %, or less than 1 wt. %, In further embodiments, the cleansing composition includes from about 0.01 to about 10 wt. %, about 0.01 to about 8 wt. %, about 0.01 to about 5 wt. %, about 0.01 to about 3 wt. %, about 0.01 to about 2 wt. %, or about 0.01 to about 1 wt. % of one or more nonionic surfactants. Preferably, the cleansing composition comprises less than 5 wt. %, more preferably less than 3 wt. %, and even more preferably less than 2 wt. % of one or more nonionic surfactants.

(c)(i) Alkanolamide Surfactants

Nonlimiting examples alkanolamide surfactants include fatty acid alkanolamide surfactants. The fatty acid alkanolamide surfactants may be fatty acid monoalkanolamides, fatty acid dialkanolamides, or fatty acid isoalkanolamides, and may have a C₂₋₈ hydroxyalkyl group (the C₂₋₈ chain can be substituted with one or more than one —OH group). Nonlimiting examples include fatty acid diethanolamides (DEA) or fatty acid monoethanolamides (MEA), fatty acid monoisopropanolamides (MIPA), fatty acid diisopropanolamides (DIPA), and fatty acid glucamides (glucamide surfactants).

Suitable fatty acid alkanolamides include those formed by reacting an alkanolamine and a C₆-C₃₆ fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and combinations thereof.

In some instances, the fatty acid alkanolamides preferably include cocamide MIPA, cocamide DEA, cocamide MEA, cocamide DIPA, and combinations thereof. In particular, the fatty acid alkanolamide may be cocamide MIPA, which is commercially available under the tradename “EMPILAN” from Innospec Active Chemicals.

Fatty acid alkanolamides surfactants include those of the following structure (XIV):

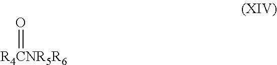

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and combinations thereof);
R₆ is selected from —CH₂OH, —CH₂CH₂OH, —CH₂CH₂CH₂OH, —CH₂(CHOH)₄CH₂OH, -benzyl, and combinations thereof;
R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and combinations thereof.

In some instances, the one or more of the fatty acid alkanolamides include one or more glucamide surfactants, for example, glucamides surfactants having a carbon chain length of 8 to 20. Nonlimiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide. Preferred glucamide surfactants include lauroyl/myristoyl methyl glucamide, cocamide methyl glucamide, lauramide methyl glucamide, myristamide methyl glucamide, caprylic/capric glucamide, oleamide methyl glucamide, stearamide methyl glucamide, behenamide methyl glucamide, palmitamide methyl glucamide, and combinations thereof.

Alkoxylated alkanolamide surfactants are a class of surfactants that combine the properties of alkanolamides with alkoxylation. Alkoxylation involves the addition of alkylene oxides (such as ethylene oxide or propylene oxide) to a chemical structure, which modifies its properties and enhances its functionality in various applications. Nonlimiting examples of alkoxylated alkanolamide surfactants include ethoxylated alkanolamides, for example, a rapeseedamide ethoxylate, a cocamide DEA ethoxylate, a cocamide MEA ethoxylate, a lauramide DEA ethoxylate, an oleamide DEA ethoxylate, a caprylic/capric acid diethanolamide ethoxylate, or combinations thereof. Preferably, the ethoxylated alkanolamide has from 2 to 100 ethoxylate groups. Further, the ethoxylated alkanolamide may have 2 to 80, 2 to 60, 2 to 50, 4 to 100, 4 to 80, 4 to 60, 4 to 50, 10 to 100, 10 to 80, 10 to 60, or 10 to 50 ethoxylate groups. Preferably, the alkanolamide is ethoxylated with 2 to 100 ethoxylate groups, more preferably with 2 to 60 ethoxylate groups, even more preferably with 2 to 40 ethoxylate groups, and even more preferably with 4 to 20 ethoxylate groups.

In a preferred embodiment, the cleansing composition includes a rapeseedamide ethoxylate, for example, one or more rapeseedamide ethoxylates selected from PEG-2 rapeseedamide, PEG-4 rapeseedamide, PEG-6 rapeseedamide, PEG-10 rapeseedamide, PEG-20 rapeseedamide, PEG-30 rapeseedamide, PEG-40 rapeseedamide, PEG-60 rapeseedamide, PEG-80 rapeseedamide.

(c)(ii) Polyglucoside Surfactants

Polyglucoside surfactants are primarily composed of glucose units linked together by glycosidic bonds. These bonds connect glucose molecules to form a polysaccharide chain, which forms the hydrophilic (water-attracting) part of the surfactant. Useful polyglucoside surfactants include those having the following formula (XV):

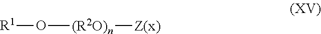

wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Nonlimiting examples of polyglucoside surfactants include decyl glucoside, lauryl glucoside, coco-glucoside, caprylyl/capryl glucoside, hexyl glucoside, octyl glucoside, decyl glucoside/coco-glucoside blend, lauryl glucoside/coco-glucoside blend, coco glucoside/benzyl alcohol, coco-glucoside/lauryl glucoside blend, lauryl glucoside/caprylyl/capryl glucoside blend, decyl glucoside/caprylyl/capryl glucoside blend, alkyl glucosides (e.g., decyl glucoside and octyl glucoside), and sugar-based glucosides such as maltodextrin-glucoside.

The cleansing composition optionally includes one or more polyglucoside surfactants. Nonetheless, in a preferred embodiment, the cleansing composition is free or essentially free from polyglucoside surfactants.

(c)(iii) Additional Nonionic Surfactants

Nonionic surfactants also include, for example, alcohols, alpha-diols, alkylphenols and esters of fatty acids, optionally being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Maltose derivatives may also be mentioned. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄) acylaminopropylmorpholine oxides; and combinations thereof.

Nonionic surfactants may be polyoxyalkylenated or polyglycerolated nonionic surfactants. The oxyalkylene units are more particularly oxyethylene or oxypropylene units, or a combination thereof, and are preferably oxyethylene units.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and combinations thereof.

Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and combinations thereof.

As glyceryl esters of fatty acids, glyceryl stearate (glyceryl mono-, di- and/or tristearate) (CTFA name: glyceryl stearate) or glyceryl ricinoleate and combinations thereof can in particular be used.

As glyceryl esters of C₈-C₂₄ alkoxylated fatty acids, polyethoxylated glyceryl stearate (glyceryl mono-, di- and/or tristearate) such as PEG-20 glyceryl stearate can for example be used.

Mixtures of these surfactants, such as for example the product containing glyceryl stearate and PEG-100 stearate, marketed under the name “ARLACEL 165” by Uniqema, and the product containing glyceryl stearate (glyceryl mono- and distearate) and potassium stearate marketed under the name “TEG1N” by Goldschmidt (CTFA name: glyceryl stearate SE), can also be used.

### (d) Water

The amount of water in the cleansing composition will vary but is typically about 60 to about 90 wt. %, based on the total weight of the cleansing composition. In further embodiments, the cleansing composition includes about 60 to about 88 wt. %, about 60 to about 85 wt. %, about 65 to about 90 wt. %, about 65 to about 88 wt. %, about 65 to about 85 wt. %, about 70 to about 90 wt. %, about 70 to about 88 wt. %, about 70 to about 85 wt. %, about 75 to about 90 wt. %, about 75 to about 88 wt. %, about 75 to about 85 wt. %, about 80 to about 90 wt. %, about 80 to about 88 wt. %, or about 80 to about 85 wt. % of water, based on the total weight of the cleansing composition. Preferably, the cleansing composition includes about 60 to about 90 wt. %, more preferably about 70 to about 88 wt. %, and even more preferably, about 75 to about 85 wt. % of water, based on the total weight of the cleansing composition.

### (e) Cationic Conditioning Polymers

The cleansing composition optionally includes one or more cationic conditioning polymers. A “cationic conditioning polymer” is a polymer having a cationic charge and provides conditioning and anti-static properties to hair. Nonlimiting examples of cationic conditioning polymers include copolymers of 1-vinyl-2-pyrrolidine and 1-vinyl-3-methyl-imidazolium salt (e.g., chloride salt) (referred to as polyquaternium-16); copolymers of 1-vinyl-2-pyrrolidine and dimethylaminoethyl methacrylate (referred to as polyquaternium-11); cationic diallyl quaternary ammonium-containing polymer including, for example, dimethyldiallyammonium chloride homopolymer and copolymers of acrylamide and dimethyldiallyammonium chloride (referred to as polyquaternium-6 and polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as polyquaternium-10). Another type of cationic cellulose includes the polymeric quaternary ammonium salts of hydroxyethyl cellulose reacted with lauryl dimethyl ammonium-substituted epoxide (referred to as polyquaternium-24). Additionally, or alternatively, the cationic conditioning polymers may include or be chosen from cationic guar gum derivatives, such as guar hydroxypropyltrimonium chloride.

In certain embodiments, the one or more cationic conditioning polymers include cationic polysaccharide polymers, such as cationic cellulose compounds, cationic starch compounds, cationic guar compounds and the like. In the context of the instant disclosure cationic polysaccharide polymers include cationic polysaccharides and polysaccharide derivatives (e.g., derivatized to be cationic), for example, resulting in cationic cellulose (cellulose derivatized to be cationic) or cationic starch (derivatized to be cationic), and the like.

Nonlimiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-4, polyquaternium-10, polyquaternium-24, and mixtures thereof.

Nonlimiting examples of cationic starch include starch hydroxypropyltrimonium chloride, hydroxypropyl oxidized starch PG trimonium chloride, and a mixture thereof.

Additional cationic conditioning polymers include quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol and the various polyquaternium types (for example 6, 7, 32 or 37) which can be found in the market under the tradenames Rheocare® CC or Ultragel® 300.

(e)(i) Cationic Guar Compounds

A cationic guar compound is a modified polysaccharide derived from guar gum, wherein the hydroxyl groups of the guar polymer have been substituted with cationic groups, such as quaternary ammonium salts. These cationic groups impart a positive charge to the polymer, allowing it to bond with negatively charged surfaces, such as those found in hair and skin, or to interact with other negatively charged components in a formulation. Nonlimiting examples of cationic guar compounds include guar hydroxypropyltrimonium chloride, guar hydroxypropyltrimethylammonium chloride (GPTC), hydroxypropyl guar hydroxypropyltrimonium chloride, guar hydroxypropyltrimethylammonium chloride, and hydroxyethylguar hydroxypropyltrimonium chloride. Preferred cationic guar compounds include hydroxypropyl guar hydroxypropyltrimonium chloride and guar hydroxypropyltrimonium chloride.

Guar hydroxypropyltrimonium chloride enhances hair manageability and softness. Guar Hydroxypropyltrimethylammonium Chloride (GPTC) is similar to guar hydroxypropyltrimonium chloride but features a quaternary ammonium group with three methyl groups. Hydroxypropyl guar hydroxypropyltrimonium chloride, which is particularly preferred, combines hydroxypropyl groups with cationic quaternary ammonium groups. Another useful cationic guar compound is guar hydroxypropyltrimethylammonium chloride, which includes trimethylammonium groups. Additionally, hydroxyethyl guar hydroxypropyltrimonium chloride, which combines hydroxyethyl groups and cationic quaternary ammonium groups, is mentioned for its conditioning and thickening properties.

(e)(ii) Polyquaterniums

The cleansing composition may include one or more polyquaterniums. Nonlimiting examples of polyquaterniums include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-4, polyquaternium-10, polyquaternium-11, polyquaternium-67, or mixture thereof.

In a preferred embodiment, the cleansing composition includes a polyquaternium selected from polyquaternium-4, polyquaternium-7, polyquaternium-10, or combinations thereof.

The total amount of the one or more cationic conditioning polymers in the cleansing composition will vary. Nonetheless, the cleansing composition typically includes from about 0.01 to about 5 wt. % of one or more cationic conditioning polymers. In further embodiments, the cleansing composition includes about 0.01 to about 3 wt. %, about 0.01 to about 2 wt. %, about 0.01 to about 1 wt. %, about 0.05 to about 5 wt. %, about 0.05 to about 3 wt. %, about 0.05 to about 2 wt. %, about 0.05 to about 1 wt. %, about 0.1 to about 5 wt. %, about 0.1 to about 3 wt. %, about 0.1 to about 2 wt. %, about 0.1 to about 1 wt. %, about 0.2 to about 5 wt. %, about 0.2 to about 3 wt. %, about 0.2 to about 2 wt. %, about 0.2 to about 1 wt. % of one or more cationic conditioning polymers, based on the total weight of the cleansing composition. Preferably, the cleansing composition includes from about 0.05 to about 3 wt. %, more preferably from about 0.1 to about 2 wt. %, and even more preferably from about 0.1 to about 1 wt. % of one or more cationic conditioning polymers.

### (f) Pearlizing Agents

The cleansing composition optionally includes one or more pearlizing agents. Pearlizing agents are used to influence a compositions opacity. Pearlizing agents are used to make the cleansing composition opaque. In various embodiments, the cleansing composition preferably includes one or more pearlizing agents and the cleansing composition is opaque.

Nonlimiting examples of pearlizing agents include pearlizing agents include glycol distearate, PEG-150 distearate, sorbitan stearate, mica, titanium dioxide, alumina, or combinations thereof. Preferred pearlizing agents include glycol distearate, PEG-150 distearate, or a combination thereof. The amount of the one or more pearlizing agents in the cleansing composition will vary but is typically about 0.05 to about 5 wt. %, based on the total weight of the cleansing composition. In further embodiments, the cleansing composition includes about 0.05 to about 3 wt. %, about 0.05 to about 2 wt. %, about 0.1 to about 5 wt. %, about 0.1 to about 3 wt. %, about 0.1 to about 2 wt. %, about 0.2 to about 5 wt. %, about 0.2 to about 3 wt. %, about 0.2 to about 2 wt. %, about 0.5 to about 5 wt. %, about 0.5 to about 3 wt. %, or about 0.5 to about 2 wt. % of one or more pearlizing agents, based on the total weight of the cleansing composition. Preferably, the amount of pearlizing agent is from about 0.05 to about 4 wt. %, more preferably from about 0.1 to about 3 wt. %, and even more preferably from about 0.2 to about 2 wt. %, based on the total weight of the cleansing composition.

### (g) Thickening Polymers

In certain embodiments, the hair treatment composition of the instant disclosure includes one or more thickening polymers. Nonlimiting examples of thickening polymers include methyl hydroxypropyl cellulose, gellan gum (such as “Kelcogel” from CP Kelco), polysaccharide, gum, hydroxyl propyl cellulose (such as “Methocel” from Dow/Amerchol), hydroxyl propyl methyl cellulose (such as “Klucel” from Hercules), hydroxyl ethyl cellulose, polyalkylene glycols, and combinations thereof. Particularly useful nonionic polymers include polysaccharide gum, hydroxyl propyl cellulose, hydroxyl propyl methyl cellulose, or combinations thereof.

The one or more thickening polymers may include polysaccharides, especially polysaccharides selected from modified or unmodified starches (such as those derived, for example, from cereals, for instance wheat, corn or rice, from vegetables, for instance yellow pea, and tubers, for instance potato or cassava), amylose, amylopectin, glycogen, dextrans, celluloses and derivatives thereof (such as methylcelluloses, hydroxyalkylcelluloses, and ethylhydroxyethylcelluloses), xylans including glucuronoxylans and arabinoxylans, glucans including xyloglucans, arabans, galactans including arabinogalactans, chitin, agars, locust bean gums, mannans including glucomannans and galactomannans such as guar gums and nonionic derivatives thereof (hydroxypropyl guar), and combinations thereof.

In a preferred embodiment, the one or more thickening polymers are selected from polyacrylate polymers, polyacrylamide polymers, carbomer, polysaccharides, gums, and a combination thereof, more preferably selected from polysaccharides, gums, and combinations thereof, and even more preferably selected from guar gums, modified guar gums such as hydroxypropyl guar, and celluloses, for example, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and combinations thereof. In some instances, the cellulose is selected from water soluble cellulose derivatives (for example, methyl cellulose, methylhydroxypropyl cellulose, cetyl hydroxyethylcellulose, hydroxyethyl cellulose, hydroxypropyl cellulose, cellulose sulfate sodium salt), and combinations thereof. Furthermore, in some instance, the cellulose is preferably cetyl hydroxyethylcellulose. Preferably, the one or more thickening polymers are nonionic thickening polymers.

The total amount of the one or more thickening polymers, if present, will vary. Nonetheless, in various embodiments, the total amount of the one or more thickening polymers is from about 0.01 to about 5 wt. %, based on the total weight of the composition. In further embodiments, the total amount of the one or more thickening polymers is from about 0.01 to about 4 wt. %, about 0.01 to about 3 wt. %, about 0.01 to about 2 wt. %, about 0.01 to about 1 wt. %, about 0.05 to about 5 wt. %, about 0.05 to about 4 wt. %, about 0.05 to about 3 wt. %, about 0.05 to about 2 wt. %, about 0.05 to about 1 wt. %, about 0.1 to about 5 wt. %, about 0.1 to about 4 wt. %, about 0.1 to about 3 wt. %, about 0.1 to about 2 wt. %, or about 0.1 to about 1 wt. %, based on the total weight of the composition.

### (h) Water Soluble Solvents

The term “water soluble solvent” is interchangeable with the term “water soluble organic solvent,” and means an organic solvent that is water soluble or can be solubilized in water and is a liquid at 25° C. and at atmospheric pressure (760 mmHg). For example, water soluble solvents may have a solubility of at least 50% in water under these conditions. Preferably, however, the water-soluble solvent have a solubility of at least 60%, 70%, 80%, 90%, or 95%.

Nonlimiting examples of water soluble solvents include glycerin, C₂-C₆ mono-alcohols, polyols, glycols, glycol ethers (for example, monomethyl, monoethyl, and monobutyl ethers of ethylene glycol or propylene glycol), propylene glycol, butylene glycol, hexylene glycol, caprylyl glycol, dipropylene glycol, alkyl ethers of diethylene glycol such as monoethyl ether or monobutyl ether of diethylene glycol (e.g., diethylene glycol monobutyl ether and diethylene glycol monoethyl ether), dipropylene glycol, and alkyl ethers of diethylene glycol, for example monoethyl ether or monobutyl ether of diethylene glycol.

Additional, nonlimiting examples of water soluble solvents include alkanediols (polyhydric alcohols or polyols) such as glycerin, 1,2,6-hexanetriol, trimethylolpropane, ethylene glycol, propylene glycol, diethylene glycol, triethylene glycol, caprylyl glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

Nonlimiting examples of polyols (polyhydric alcohols) include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a combination thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, of combinations thereof.

Glycols are a group of chemical compounds that belong to the alcohol family, specifically diols, characterized by having two hydroxyl (—OH) groups attached to different, adjacent carbon atoms within the same molecule. Nonlimiting examples of glycols having from 2 to 10 carbon atoms include ethylene glycol, propylene glycol, propanediol (propane-1,3-diol), butylene glycol, hexylene glycol, caprylyl glycol, pentylene glycol, propanediol, and diethylene glycol. Preferred glycols include propylene glycol, propanediol, butylene glycol, diethylene glycol, and combinations thereof. Propanediol is particularly preferred.

In various embodiments, the cleansing composition includes one or more water soluble solvents selected from glycerin, C₂-C₆ mono-alcohols, polyols, glycols, or a combinations thereof, for example, ethanol, isopropanol, t-butyl alcohol, ethylene glycol, propylene glycol, butylene glycol, pentylene glycol, dipropylene glycol, glycerin, or combinations thereof. In a preferred embodiment, one of the one or more water soluble solvents is ethanol.

The total amount of water-soluble solvents in the cleansing composition, if present, can vary but is typically from about 0.01 to about 10 wt. %, based on the total weight of the cleansing composition. In further embodiments, the cleansing composition includes about 0.01 to about 8 wt. %, about 0.01 to about 5 wt. %, about 0.01 to about 3 wt. %, about 0.01 to about 2 wt. %, about 0.01 to about 1 wt. % of one or more water soluble solvents, based on the total weight of the cleansing composition. Preferably, the cleansing composition includes less than 5 wt. %, more preferably less than 3 wt. %, even more preferably less than 2 wt. %, and even more preferably less than 1 wt. % of C₂-C₆ mono-alcohols, glycerin, and glycols having from 2 to 10 carbon atoms.

In a preferred embodiment, the cleansing composition comprises less than 5 wt. % of C₂-C₆ mono-alcohols, glycerin, and glycols having from 2 to 10 carbon atoms. In further embodiments, the cleansing composition comprises less than 4 wt. %, less than 3 wt. %, less than 2 wt. %, less than 1 wt. %, less than 0.5 wt. %, or less than 0.1 wt. % of C₂-C₆ mono-alcohols, glycerin, and glycols having from 2 to 10 carbon atoms. Similarly, the cleansing composition my preferably be free from of C₂-C₆ mono-alcohols, glycerin, and glycols having from 2 to 10 carbon atoms.

### (i) Silicones

The cleansing composition may optionally include one or more silicones. Nonlimiting examples of silicones include dimethicones, dimethiconols, and aminosilicones. Preferably, the one or more silicones are amino-silicones. The term “amino-silicone” or “amino-functionalized silicone” means a silicone containing at least one primary amino, secondary amino, tertiary amino and/or quaternary ammonium group. The structure of the amino-silicone may be linear or branched, cyclic or non-cyclic. The amino functional group may be at any position in the silicone molecule, preferably at the end of the backbone (for example, in the case of amodimethicones) and/or in the side chain. Nonlimiting examples of amino-silicones include bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH3 in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename DOWSIL® AP-8087 FLUID from The Dow Chemical Company. A particularly preferred amino-silicone is amodimethicone, for example, sold by Wacker under the name BELSIL® ADM 652, BELSIL® ADM 4000 E, or BELSIL® ADM LOG 1. Additionally or alternative, the weight-average molecular weight (MW) of the silicone preferably ranges from 2,000 to 200,000, even more preferably 5,000 to 100,000 and more preferably from 10,000 to 50,000.

In a preferred embodiment, the one or more amino-silicones are selected from amodimethicone, bis-hydroxy/methoxy amodimethicone, bis-cetearyl amodimethicone, bis(C13-15 alkoxy) PG amodimethicone, aminopropyl phenyl trimethicone, aminopropyl dimethicone, bis-amino PEG/PPG-41/3 aminoethyl PG-propyl dimethicone, or a mixture thereof. In a further preferred embodiment, the amino-silicone is amodimethicone.

The total amount of the one or more silicones in the cleansing composition, if present, will vary but is typically from about 0.01 to about 3 wt. %, based on the total weight of the cleansing composition. In further embodiments, the cleansing composition includes from about 0.01 to about 2 wt. %, about 0.01 to about 1 wt. %, about 0.01 to about 0.5 wt. %, about 0.05 to about 3 wt. %, about 0.05 to about 2 wt. %, about 0.05 to about 1 wt. %, about 0.1 to about 3 wt. %, about 0.1 to about 2 wt. %, about 0.1 to about 1 wt. %, or about 0.1 to about 0.5 wt. %, based on the total weight of the cleansing composition. If present, the one or more silicones is preferably in an amount from about 0.01 to about 2 wt. %, more preferably about 0.05 to about 1 wt. %, and even more preferably about 0.1 to about 0.5 wt. %, based on the total weight of the cleansing composition.

### (j) Direct Dyes

Nonlimiting examples of direct dyes generally include azo direct dyes; anthraquinone and anthraquinone derivatives; (poly) methine dyes such as cyanins, hemicyanins and styryls; carbonyl dyes; azine dyes; nitro (hetero) aryl dyes; tri(hetero)arylmethane dyes; porphyrin dyes; phthalocyanin dyes, and natural direct dyes, alone or as mixtures. The direct dyes may be cationic or anionic.

Non-limiting examples of cationic dyes include Basic Blue 6, Basic Blue 7, Basic Blue 9, Basic Blue 26, Basic Blue 41, Basic Blue 99, Basic Brown 4, Basic Brown 16, Basic Brown 17, Natural Brown 7, Basic Green, Basic Orange 31, 1, Basic Red 2, Basic Red 12 Basic Red 22, Basic Red 76 Basic Red 51, Basic Violet 1, Basic Violet 2, Basic Violet 3, Basic Violet 10, Basic Violet 14, Basic Yellow 57 and Basic Yellow 87.

Non-limiting examples anionic dyes include Acid Black 1, Acid Blue 1, Acid Blue 3, Food Blue 5, Acid Blue 7, Acid Blue 9, Acid Blue 74, Acid Orange 3, Acid Orange 6, Acid Orange 7, Acid Orange 10, Acid Red 1, Acid Red 14, Acid Red 18, Acid Red 27, Acid Red 50, Acid Red 52, Acid Red 73, Acid Red 87, Acid Red 88, Acid Red 92, Acid Red 155, Acid Red 180, Acid Violet 9, Acid Violet 43, Acid Violet 49, Acid Yellow 1, Acid Yellow 23, Acid Yellow 3, Food Yellow No. 8, D&C Brown No. 1, D&C Green No. 5, D&C Green No. 8, D&C Orange No. 4, D&C Orange No. 10, D&C Orange No. 11, D&C Red No. 21, D&C Red No. 27, D&C Red No. 33, D&C Violet 2 (Ext Violet 2), D&C Yellow No. 7, D&C Yellow No. 8, D&C Yellow No. 10, FD&C Red 2, FD&C Red 40, FD&C Red No. 4, FD&C Yellow No. 6, FD&C Blue 1, Food Black 1, Food Black 2, Disperse Black 9 and Disperse Violet 1 and their alkali metal salts such as sodium and/or potassium.

Non-limiting examples of nitro dyes include HC Blue No. 2, HC Blue No. 4, HC Blue No. 5, HC Blue No. 6, HC Blue No. 7, HC Blue No. 8, HC Blue No. 9, HC Blue No. 10, HC Blue No. 11, HC Blue No. 12, HC Blue No. 13, HC Blue No. 17, HC Brown No. 1, HC Brown No. 2, HC Green No. 1, HC Orange No. 1, HC Orange No. 2, HC Orange No. 3, HC Orange No. 5, HC Red BN, HC Red No. 1, HC Red No. 3, HC Red No. 7, HC Red No. 8, HC Red No. 9, HC Red No. 10, HC Red No. 11, HC Red No. 13, HC Red No. 54, HC Red No. 14, HC Violet BS, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, 2-Amino-6-chloro-4-nitrophenol, picramic acid, 1,2-Diamino-4-nitrobenzol, 1,4-Diamino-2-nitrobenzol, 3-Nitro-4-aminophenol, 1-Hydroxy-2-amino-3-nitrobenzol and 2-hydroxyethylpicramic acid.

The total amount of the one or more direct dyes in the cleansing composition, if present will vary but is typically from about 0.001 to about 3 wt. % based on the total weight of the developer composition. In further embodiments, the total amount of the one or more direct dyes co is from about 0.001 to about 3 wt. %, about 0.001 to about 2 wt. %, about 0.001 to about 1 wt. %, about 0.001 to about 0.5 wt. %, about 0.005 to about 3 wt. %, about 0.005 to about 2 wt. %, about 0.005 to about 1 wt. %, about 0.005 to about 0.5 wt. %, about 0.01 to about 3 wt. %, about 0.01 to about 2 wt. %, about 0.01 to about 1 wt. %, about 0.01 to about 0.5 wt. %, based on the total weight of the composition. Preferably, if present, the cleansing composition comprises from about 0.001 to about 2 wt. %, more preferably about 0.01 to about 1 wt. %, and even more preferably about 0.05 to about 2 wt. % of the one or more direct dyes.

### (k) Miscellaneous Ingredients

The cleansing compositions may optionally include one or more miscellaneous ingredients. Miscellaneous ingredients are ingredients that are compatible with the cleansing composition and do not disrupt or materially affect the basic and novel properties of the composition. Nonlimiting examples of miscellaneous ingredients include preservatives, fragrances, pH adjusters, salts, chelating agents, amino acids (e.g., taurine), buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, particular materials, etc.) composition colorants, etc. In various embodiments, the miscellaneous ingredients are chosen from preservatives, fragrances, pH adjusters, salts, chelating agents, buffers, composition colorants, and vegetal or botanic extracts.

In various embodiments, at least one of the one or more miscellaneous ingredients a salt. Salts can thicken shampoo by interacting with the surfactant molecules and lowering the charge density on the outside of micelles. Nonlimiting examples include sodium chloride and potassium chloride, preferably sodium chloride. The salt is typically in amounts from about 0.1 to about 3 wt. %, based on the total weight of the cleansing composition. Preferably, the salt is in an amount of about 0.1 to about 2 wt. %, more preferably about 0.2 to about 1.5 wt. %, and even more preferably about 0.3 to about 1.3 wt. %, based on the total weight of the cleansing composition.

Nonlimiting examples of pH adjusters include citric acid, lactic acid, sodium hydroxide, potassium hydroxide, ammonium hydroxide, sodium citrate, sodium bicarbonate, phosphoric acid, acetic acid, and the like.

In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair. Further, for purposes of the instant disclosure, pearlescent or pearlizing agents are considered composition colorants. Pearlescent or pearlizing agents are cosmetic ingredients primarily used to give a shimmering effect or pearlescent effect to the composition. They can be used to enhance the visual appeal of the cleansing composition.

In addition to the above, the components described as optional components throughout the disclosure are can also be considered miscellaneous ingredients if not expressly set forth as an independent component of the cleansing composition. For example, if a particular component is not expressly set forth in a claim that expressly allows for miscellaneous ingredients, such optional components should be considered included in the miscellaneous ingredients unless otherwise specified. Thus, the term “miscellaneous ingredient” is understood as a catch-all phrase representing additional components that may optionally be present in amounts designated for the miscellaneous ingredients.

The cleansing composition optionally includes salicylic acid. Salicylic acid helps to remove scalp buildup, treat dandruff and seborrheic dermatitis, control oily scalp, and promote healthy scalp and hair. Salicylic acid may be included as one of the one or more miscellaneous ingredients or may be specifically included as individual component of the cleansing composition. If present, the salicylic acid is typically in an amount of about 0.01 to about 2 wt. %, based on the total weight of the cleansing composition. Preferably, if present, the salicylic acid is present in an amount from about 0.05 to about 2 wt. %, more preferably from about 0.1 to about 1.5 wt. %, and even more preferably from about 0.1 to about 1 wt. %, based on the total weight of the hair cleansing composition.

The amount of the one or more miscellaneous ingredients in the cleansing composition, if present, will vary but is typically from about 0.01 to about 10 wt. %, based on the total weight of the cleansing composition. In further embodiments, the cleansing composition includes about 0.01 to about 8 wt. %, about 0.01 to about 6 wt. %, about 0.01 to about 5 wt. %, about 0.01 to about 3 wt. %, about 0.05 to about 10 wt. %, about 0.05 to about 8 wt. %, about 0.05 to about 6 wt. %, about 0.05 to about 5 wt. %, about 0.1 to about 10 wt. %, about 0.1 to about 8 wt. %, about 0.1 to about 6 wt. %, about 0.1 to about 5 wt. % of one or more miscellaneous ingredients, based on a total weight of the cleansing composition.

pH

The pH of the cleansing composition will vary but is typically from about 4 to less than 7. Preferably, the pH of the cleansing composition is about 4 to about 6.5, about 4 to about 6.2, about 4.5 to less than 7, about 4.5 to about 6.5, about 4.5 to about 6.2, about 5 to less than 7, about 5 to about 6.5, about 5 to about 6.2, about 5.5 to less than 7, about 5.5 to about 6.5, or about 5.5 to about 6.2. Preferably, the pH of the cleansing composition is from about 4.5 to about 6.8, more preferably from about 5 to about 6.5, and even more preferably from about 5.5 to about 6.2.

### Viscosity

The viscosity of the cleansing composition will vary but is typically from about 500 to about 30,000 cPs at 25° C. In further embodiments, the viscosity of the cleansing composition may be from about 500 to about 25,000 cPs, about 500 to about 20,000 cPs, about 500 to about 10,000 cPs, about 500 to about 12,000 cPs, about 500 to about 10,000 cPs, about 1,000 to about 30,000 cPs, about 1,000 to about 25,000 cPs, about 1,000 to about 20,000 cPs, about 1,000 to about 15,000 cPs, about 1,000 to about 15,000 cPs, about 1,000 to about 12,000 cPs, about 1,000 to about 10,000 cPs, about 2,000 to about 30,000 cPs, about 2,000 to about 25,000 cPs, about 2,000 to about 20,000 cPs, about 2,000 to about 15,000 cPs, about 2,000 to about 12,000 cPs, about 2,000 to about 10,000 cPs, about 5,000 to about 25,000 cPs, about 5,000 to about 20,000 cPs, about 5,000 to about 15,000 cPs, about 5,000 to about 12,000 cPs, or about 5,000 to about 10,000 cPs at 25° C. The viscosity can be determined using a Brookfield DV-1 viscometer with a spindle #RV05, at 12 RPM, at 25° C. The viscosity can also be measure using a Rheomat rheometer, for example with a Spindle 2, at 25° C. Preferably the viscosity is from about 2,000 to about 25,000 cPs, more preferably from about 2,000 to about 20,000 cPs, and more preferably from about 5,000 to about 15,000 cPs at 25° C.

In various embodiments, the viscosity of the cleansing composition is substantially maintained for long periods of time, for example at least 1 week, at least 1 month (4 weeks), at least 2 months (8 weeks), or at least 3 months (12 weeks). More specifically, the cleansing composition does not lose more than 15% of its viscosity after storage at 25° C. for at least 1 month (4 weeks), at least 2 months (8 weeks), or at least 3 months (12 weeks). More preferably, the cleansing composition does not lose more than 10%, and even more preferably more than 5% of its viscosity after storage at 25° C. for at least 1 month (4 weeks), at least 2 months (8 weeks), or at least 3 months (12 weeks).

### Stability

The cleansing compositions may be subjected to freeze-thaw stability studies and the stability of the composition visually assessed for particulate formation and phase separation. The stability of the compositions are visually accessed upon initial manufacture (To). The compositions are again evaluated after one week of freeze-thaw testing (1W FT). During freeze-thaw testing, the compositions are placed in a stability chamber and subjected to temperature fluctuation at 12-hour intervals. For 12 hours, the compositions are held at −10° C. For the next 12 hours, the compositions are held at 25° C. The cycle is repeated 7 times (for 1 week). After the week of freeze-thaw testing, the compositions are visually assessed for particulate formation and phase separation.

In various embodiments, the cleansing composition does not form visible particulates or phase separate after one week of freeze-thaw testing.

The compositions may separately be evaluated, with freeze-thaw steps, after storage at 4° C. and after storage at 45° C., for example at 1 week, at 1 month (4 weeks), at 2 months (8 weeks), and at 3 months (12 weeks). After storage, the compositions are visually assessed for particulate formation and phase separation.

In various embodiments, the cleansing composition does not form visible particulate or phase separate at 1 week, at 1 month (4 weeks), at 2 months (8 weeks), and/or at 3 months (12 weeks) after storage at 4° C., 25° C., 37° C., and/or 45° C.

### Optional Exclusions

As mentioned earlier, the cleansing composition is free from silicones and sulfate-based surfactants. In addition, however, the cleansing composition is optionally free or essentially free from other components. For example, in various embodiments, the cleansing composition is preferably free or essentially free from cationic surfactants.

The cleansing composition may be free or essentially from glycerin and/or glycols having from having from 2 to 10 carbon atoms.

The cleansing composition may be free or essentially free from fatty acids. In various embodiments, the cleansing composition comprises less than 5 wt. % of one or more fatty acids, based on the total weight of the cleansing composition. Preferably, however, the cleansing composition comprises less than 5 wt. %, preferably less than 3 wt. %, and more preferably less than 2 wt. % of fatty acids.

The cleansing composition may be free or essentially free from fatty alcohols. In various embodiments, the cleansing composition comprises less than 5 wt. % of one or more fatty alcohols, based on the total weight of the cleansing composition. Preferably, however, the cleansing composition comprises less than 5 wt. %, less than 3 wt. %, and more preferably less than 2 wt. % of fatty alcohols. In other embodiments, the cleansing composition preferably includes from about 0.1 to about 5 wt. %, preferably from about 0.5 to about 4 wt. %, and more preferably from about 1 to about 3 wt. % of one or more fatty alcohols.

In various embodiments, the hair cleansing composition is free or essentially free from fatty compounds, especially oils. Fatty compounds are a diverse group of organic molecules characterized by their insolubility in water (hydrophobic nature) and solubility in nonpolar solvents. Nonlimiting examples of fatty compounds include fatty acids, fatty alcohols, triglycerides, phospholipids, steroids, waxes, oils, glycerides, sphinoglipids, eisosanoids, alkylglycerols, polyketides, fatty acid amides, and ether lipids. In various embodiments, the cleansing composition comprises less than 5 wt. % of one or more fatty compounds, based on the total weight of the cleansing composition. Preferably, however, the cleansing composition comprises less than 3 wt. %, more preferably less than 2 wt. %, even more preferably less than 1 wt. %, and even more preferably less than 0.5 wt. % of fatty compounds.

In a preferred embodiment, the cleansing composition is free or essentially free from polyglucoside surfactants. In various embodiments, the cleansing composition comprises less than 5 wt. % of one or more polyglucoside surfactants, based on the total weight of the cleansing composition. Preferably, however, the cleansing composition comprises less than 3 wt. %, more preferably less than 2 wt. %, even more preferably less than 1 wt. %, and even more preferably less than 0.5 wt. % of polyglucoside surfactants.

In various embodiments, the cleansing composition is free or essentially free from oils. In the context of the present disclosure, the term “oil” refers to a liquid substance that is hydrophobic and may include a mixture of hydrocarbons, fatty acids, esters, or other organic compounds. Oils can be derived from various sources including, but not limited to, natural vegetable or animal fats, synthetic chemical processes, or mineral deposits. They are characterized by their ability to remain in a liquid state at room temperature (25° C.). In various embodiments, the cleansing composition includes less than 5 wt. % of oils, based on the total weight of the cleansing composition. Preferably, however, the cleansing composition comprises less than 3 wt. %, more preferably less than 2 wt. %, even more preferably less than 1 wt. %, and even more preferably less than 0.5 wt. % of oils.

In various embodiments, the cleansing composition is preferably free or essentially free from cationic proteins and cationic protein hydrolysates, for example, hydroxypropyltrimonium hydrolyzed wheat protein.

### Methods

The cleansing compositions are particularly useful for cleansing and conditioning hair. Additionally, the cleansing compositions provide a variety of desirable cosmetic and styling benefits to the hair, for example, smoothness, detangling, and shine. Accordingly, the cleansing compositions are useful in methods for cleansing hair, methods for conditioning hair, and methods for imparting smoothness, detangling, and/or shine to hair. Such methods include, for example, applying the cleansing composition to the hair, preferably wet or damp hair, working the composition throughout the hair, and rinsing the composition from the hair.

Methods for cleansing and conditioning the hair include applying an effective amount of a cleansing composition of the hair, allowing the cleansing composition to remain on the hair for a period of time, and subsequently rinsing the cleansing composition from the hair. Usually, the cleansing composition is merely allowed to remain on the hair for a period of time sufficient to incorporate the cleansing composition throughout the hair, for example, by lathering the composition throughout the hair using one's hands. As is common when using shampoo and/or conditioning compositions, the hair may be wetted or rinsed with water prior to application of a cleansing composition of the instant disclosure. Having water already in the hair can be helpful for creating lather when applying the cleansing compositions because the water interacts with the surfactants of the surfactant system. The cleansing composition is subsequently rinsed from the hair.

### PREFERRED EMBODIMENTS

In a preferred embodiment, the cleansing composition comprises, consists essentially of, or consists of:
(a) about 2 to about 20 wt. %, preferably about 3 to about 15 wt. %, more preferably about 4 to about 10 wt. % of a plurality of non-sulfate anionic surfactants, the plurality of non-sulfate anionic surfactants comprising, consisting essentially of, or consisting of:
   (i) about 1 to about 12 wt. %, preferably about 2 to about 10 wt. %, more preferably about 3 to about 8 wt. % of one or more isethionate surfactants, wherein the one or more isethionate surfactants are preferably selected from sodium cocoyl isethionate, sodium lauroyl isethionate, sodium myristoyl isethionate, sodium capryloyl isethionate, sodium stearoyl isethionate, sodium oleoyl isethionate, sodium palmitoyl isethionate, sodium lauroyl methyl isethionate, sodium cocoyl methyl isethionate, sodium decanoyl isethionate, sodium lauroyl sarcosinate, sodium cetyl isethionate, sodium isethionate, sodium cocoate isethionate, sodium hexanoyl isethionate, sodium tetradecanoyl isethionate, sodium nonanoyl isethionate, sodium oleoyl methyl isethionate, sodium isethionate laurate, sodium isethionate stearate, sodium dodecanoyl isethionate, sodium palmitoleoyl isethionate, sodium isethionate caprate, sodium isethionate linoleate, sodium lauryl isethionate, sodium caproyl isethionate, and sodium isethionate behenate, wherein sodium cocoyl isethionate is particularly preferred;
   (ii) about 0.1 to about 12 wt. %, preferably about 0.5 to about 5 wt. %, more preferably about 1 to about 3 wt. % of one or more amino acid surfactants, wherein preferably, the one or more amino acid surfactants are selected from sarcosinate surfactants, taurate surfactants, glycinate surfactants, and glutamate surfactants, wherein more preferably, the one or more amino acid surfactants comprises or consists of one or more sarcosinate surfactants, one or more taurate surfactants, or a combination thereof;
   (iii) about 0.1 to about 5 wt. %, preferably about 0.2 to about 4 wt. %, more preferably about 0.5 to about 3 wt. % of one or more alkoxylated monoacid surfactants, preferably wherein the one or more alkoxylated monoacid surfactants are ethoxylated with from 2 to 20 ethoxylate groups;
      wherein (a) (ii) and (a) (iii) are preferably in a combined amounts of about 0.2 to less than 5 wt. %, more preferably about 0.5 to about 4 wt. %, and even more preferably about 0.5 to about 3 wt. %; and
   (iv) optionally, about 0.01 to about 10 wt. %, about 0.05 to about 6 wt. %, or about 0.1 to about 5 wt. % of one or more additional non-sulfate anionic surfactants;
(b) about 1 to about 15 wt. %, preferably about 2 to about 12 wt. %, more preferably about 5 to about 10 wt. % of one or more amphoteric surfactants, wherein preferably, the one or more amphoteric surfactants are selected from betaine surfactants, sultaine surfactants, amphopropionate surfactants, and amphoacetate surfactants, wherein even more preferably, the one or more amphoteric surfactants comprise or consist of betaine surfactants;
(c) optionally, one or more nonionic surfactants, wherein preferably, the cleansing composition comprises less than 5 wt. % of one or more nonionic surfactants, more preferably, the cleansing composition comprises less than 2 wt. % of one or more nonionic surfactants:
(d) about 60 to about 90 wt. %, preferably about 65 to about 88 wt. %, more preferably about 70 to about 85 wt. % of water;
(e) optionally, about 0.1 to about 5 wt. %, preferably about 0.1 to about 4 wt. %, more preferably about 0.2 to about 2 wt. % of one or more cationic conditioning polymers, wherein the one or more cationic guar compounds are preferably selected from cationic guar compounds, polyquaterniums, or combinations thereof, wherein the one or more cationic guar compounds are preferably selected from guar hydroxypropyltrimonium chloride, guar hydroxypropyltrimethylammonium chloride (GPTC), hydroxypropyl guar hydroxypropyltrimonium chloride, guar hydroxypropyltrimethylammonium chloride, and hydroxyethylguar hydroxypropyltrimonium chloride and the one or more polyquaterniums are preferably selected from polyquaternium-4, polyquaternium-7, polyquaternium-10, and combinations thereof;
(f) optionally, about 0.05 to about 4 wt. %, preferably about 0.1 to about 3 wt. %, and more preferably about 0.5 to about 2 wt. % of one or more pearlizing agents, wherein the one or more pearlizing agents are preferably selected from glycol distearate, PEG-150 distearate, sorbitan stearate, mica, titanium dioxide, alumina, or combinations thereof, more preferably wherein the pearlizing agent is glycol distearate, PEG-150 distearate, or a combination thereof;
(g) optionally, about 0.001 to about 5 wt. %, preferably about 0.01 to about 3 wt. %, more preferably about 0.1 to about 2 wt. % of one or more direct dyes;
(h) optionally, about 0.1 to about 10 wt. % of one or more miscellaneous ingredients, wherein the one or more miscellaneous ingredients are preferably selected from pH adjusters, salts, fragrances, preservatives, antioxidants, skin active agents, anti-wrinkle agent, UV filters, de-pigmenting agents, chelating agents, compositions colorants, pearlizing agents, fillers, humectants, emollients, botanical extracts, amino acids, peptides, proteins, or a combination thereof;
   wherein the composition is free from sulfate-based anionic surfactants;
   wherein preferably, the composition has a pH of about 4 to less than 7, more preferably about 4.5 to about 6.5, and even more preferably about 5.5 to about 6.2;
   wherein optionally, the composition preferably comprises less than 2 wt. %, more preferably less than 1 wt. %, and even more preferably less than 0.5 wt. % of C1-C6 monoalcohols and
   all percentages by weight are based on the total weight of the cleansing composition.

In another preferred embodiments, the cleansing composition comprises, consists essentially of, or consists of:
(a) about 2 to about 20 wt. %, preferably about 3 to about 15 wt. %, more preferably about 4 to about 12 wt. % of a plurality of non-sulfate anionic surfactants, the plurality of non-sulfate anionic surfactants comprising, consisting essentially of, or consisting of:
   (i) about 1 to about 12 wt. %, preferably about 2 to about 10 wt. %, more preferably about 3 to about 8 wt. % of one or more isethionate surfactants, wherein the one or more isethionate surfactants are preferably selected from sodium cocoyl isethionate, sodium lauroyl isethionate, sodium myristoyl isethionate, sodium capryloyl isethionate, sodium stearoyl isethionate, sodium oleoyl isethionate, sodium palmitoyl isethionate, sodium lauroyl methyl isethionate, sodium cocoyl methyl isethionate, sodium decanoyl isethionate, sodium lauroyl sarcosinate, sodium cetyl isethionate, sodium isethionate, sodium cocoate isethionate, sodium hexanoyl isethionate, sodium tetradecanoyl isethionate, sodium nonanoyl isethionate, sodium oleoyl methyl isethionate, sodium isethionate laurate, sodium isethionate stearate, sodium dodecanoyl isethionate, sodium palmitoleoyl isethionate, sodium isethionate caprate, sodium isethionate linoleate, sodium lauryl isethionate, sodium caproyl isethionate, and sodium isethionate behenate, and even more preferably, the one or more isethionate surfactants comprises or consists of sodium cocoyl isethionate;
   (ii) about 0.5 to about 12 wt. %, preferably about 1 to about 10 wt. %, more preferably about 1 to about 5 wt. % of one or more amino acid surfactants, wherein preferably, the one or more amino acid surfactants are selected from sarcosinate surfactants, taurate surfactants, glycinate surfactants, and glutamate surfactants, wherein more preferably, the one or more amino acid surfactants comprises or consists of a sarcosinate surfactants selected from sodium lauroyl sarcosinate, sodium cocoyl sarcosinate, sodium myristoyl sarcosinate, sodium palmitoyl sarcosinate, sodium stearoyl sarcosinate, sodium oleoyl sarcosinate, sodium linoleoyl sarcosinate, sodium linolenoyl sarcosinate, sodium capryloyl sarcosinate, sodium caproyl sarcosinate, sodium lauric acid sarcosinate, sodium lauroyl methyl sarcosinate, sodium cocoyl methyl sarcosinate, sodium myristoyl methyl sarcosinate, sodium palmitoyl methyl sarcosinate, sodium stearoyl methyl sarcosinate, sodium oleoyl methyl sarcosinate, sodium linoleoyl methyl sarcosinate, sodium linolenoyl methyl sarcosinate, sodium capryloyl methyl sarcosinate, sodium caproyl methyl sarcosinate, sodium lauryl sarcosinate, sodium cocoyl sarcosinate, sodium myristyl sarcosinate, sodium palmityl sarcosinate, sodium stearyl sarcosinate, sodium oleyl sarcosinate, sodium linoleyl sarcosinate, sodium linolenyl sarcosinate, sodium caprylyl sarcosinate, and sodium caprolyl sarcosinate, wherein even more preferably, the one or more sarcosinate surfactants comprises or consists of sodium lauroyl sarcosinate; and
   (iii) about 0.1 to about 5 wt. %, preferably about 0.2 to about 4 wt. %, more preferably about 0.5 to about 3 wt. % of one or more alkoxylated monoacid surfactants, preferably wherein the one or more alkoxylated monoacid surfactants are ethoxylated with from 2 to 20 ethoxylate groups, even more preferably the one or more alkoxylated monoacid surfactants are selected from trideceth-7 carboxylic acid, sodium laureth-13 carboxylate, sodium laureth-4 carboxylate, laureth-11 carboxylic acid, laureth-5 carboxylic acid, sodium laureth-5 carboxylate, ammonium laureth-6 carboxylate, ammonium laureth-8 carboxylate, capryleth-4 carboxylic acid, capryleth-6 carboxylic acid, capryleth-9 carboxylic acid, ceteareth-25 carboxylic acid, cetyl c12-15 pareth-8 carboxylate, cetyl C12-15 pareth-9 carboxylate, cetyl ppg-2 isodeceth-7 carboxylate, coceth-7 carboxylic acid, C9-11 pareth-6 carboxylic acid, C9-11 pareth-8 carboxylic acid, C11-15 pareth-7 carboxylic acid, C12-13 pareth-5 carboxylic acid, C12-13 pareth-7 carboxylic acid, C12-13 pareth-8 carboxylic acid, C12-13 pareth-12 carboxylic acid, C12-15 pareth-7 carboxylic acid, C12-15 pareth-8 carboxylic acid, C12-15 pareth-12 carboxylic acid, C14-15 pareth-8 carboxylic acid, deceth-7 carboxylic acid, ethylhexeth-3 carboxylic acid, hexeth-4 carboxylic acid, isopropyl C12-15-pareth-9 carboxylate, isopropyl ppg-2 isodeceth-7 carboxylate, isosteareth-6 carboxylic acid, isosteareth-11 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, magnesium laureth-11 carboxylate, mea-laureth-6 carboxylate, mea ppg-6 laureth-7 carboxylate, mea-ppg-8-stearath-7 carboxylate, myreth-3 carboxylic acid, myreth-5 carboxylic acid, oleth-3 carboxylic acid, oleth-6 carboxylic acid, oleth-10 carboxylic acid, peg-2 stearamides carboxylic acid, peg-9 stearamide carboxylic acid, potassium laureth-3 carboxylate, potassium laureth-4 carboxylate, potassium laureth-5 carboxylate, potassium laureth-6 carboxylate, potassium laureth-10 carboxylate, potassium trideceth-3 carboxylate, potassium trideceth-4 carboxylate, potassium trideceth-7 carboxylate, potassium trideceth-15 carboxylate, potassium trideceth-19 carboxylate, ppg-3-deceth-2 carboxylic acid, propyl c12-15 pareth-8 carboxylate, sodium capryleth-2 carboxylate, sodium capryleth-9 carboxylate, sodium ceteareth-13 carboxylate, sodium ceteth-13 carboxylate, sodium C9-11 pareth-6 carboxylate, sodium C11-15 pareth-7 carboxylate, sodium C12-13 pareth-5 carboxylate, sodium C12-13 pareth-8 carboxylate, sodium C12-13 pareth-12 carboxylate, sodium C12-15 pareth-6 carboxylate, sodium C12-15 pareth-7 carboxylate, sodium C12-15 pareth-8 carboxylate, sodium C12-15 pareth-12 carboxylate, sodium C14-15 pareth-8 carboxylate, sodium C12-14 sec-pareth-8 carboxylate, sodium deceth-2 carboxylate, sodium hexeth-4 carboxylate, sodium isosteareth-6 carboxylate, sodium isosteareth-11 carboxylate, sodium laureth-3 carboxylate, sodium laureth-4 carboxylate, sodium laureth-5 carboxylate, sodium laureth-6 carboxylate, sodium laureth-8 carboxylate, sodium laureth-11 carboxylate, sodium laureth-12 carboxylate, sodium laureth-13 carboxylate, sodium laureth-14 carboxylate, sodium laureth-16 carboxylate, sodium laureth-17 carboxylate, sodium lauryl glucose carboxylate, sodium lauryl glycol carboxylate, sodium peg-6 cocamide carboxylate, sodium peg-8 cocamide carboxylate, sodium peg-3 lauramide carboxylate, sodium peg-4 lauramide carboxylate, sodium peg-7 olive oil carboxylate, sodium peg-8 palm glycerides carboxylate, sodium trideceth-3 carboxylate, sodium trideceth-4 carboxylate, sodium trideceth-6 carboxylate, sodium trideceth-7 carboxylate, sodium trideceth-8 carboxylate, sodium trideceth-12 carboxylate, sodium trideceth-15 carboxylate, sodium trideceth-19 carboxylate, sodium undeceth-5 carboxylate, trideceth-3 carboxylic acid, trideceth-4 carboxylic acid, trideceth-7 carboxylic acid, trideceth-8 carboxylic acid, trideceth-15 carboxylic acid, trideceth-19 carboxylic acid and undeceth-5 carboxylic acid;
      wherein (a) (ii) and (a) (iii) are preferably in a combined amounts of about 0.2 to less than 5 wt. %, more preferably about 0.5 to about 4 wt. %, and even more preferably about 0.5 to about 3 wt. %; and
   (iv) optionally, about 0.01 to about 10 wt. %, about 0.05 to about 6 wt. %, or about 0.1 to about 5 wt. % of one or more additional non-sulfate anionic surfactants;
(b) about 1 to about 20 wt. %, preferably about 2 to about 15 wt. %, more preferably about 3 to about 12 wt. %, and even more preferably about 5 to about 10 wt. % of one or more amphoteric surfactants, wherein preferably, the one or more amphoteric surfactants are selected from betaine surfactants, sultaine surfactants, amphopropionate surfactants, and amphoacetate surfactants, wherein even more preferably, the one or more amphoteric surfactants comprise or consist of betaine surfactants, wherein even more preferably, the one or more betaine surfactants are selected from coco betaine, cocamidopropyl betaine, lauryl betaine, laurylhydroxy sulfobetaine, lauryldimethyl betaine, behenyl betaine, capryl/capramidopropyl betaine, stearyl betaine, lauramidopropyl betaine, myristamidopropyl betaine, palmitamidopropyl betaine, stearamidopropyl betaine, oleamidopropyl betaine, capramidopropyl betaine, caprylamidopropyl betaine, behenamidopropyl betaine, lauramidopropyl betaine, cocamidopropyl hydroxysultaine, and cocamidopropyl dimethylamine betaine;
(c) less than 5 wt. %, more preferably less than 3 wt. %, and more preferably less than 2 wt. % of one or more nonionic surfactants, wherein preferably, the cleansing compositions is free or essentially free from polyglucoside surfactants:
(d) about 60 to about 90 wt. %, preferably about 65 to about 88 wt. %, more preferably about 70 to about 85 wt. % of water;
(e) optionally, about 0.1 to about 5 wt. %, preferably about 0.1 to about 4 wt. %, more preferably about 0.2 to about 2 wt. % of one or more, preferably two or more, cationic conditioning polymers, wherein the one or more (or preferably two or more) cationic guar compounds are selected from cationic guar compounds, polyquaterniums, or combinations thereof and the one or more cationic guar compounds are selected from guar hydroxypropyltrimonium chloride, guar hydroxypropyltrimethylammonium chloride (GPTC), hydroxypropyl guar hydroxypropyltrimonium chloride, guar hydroxypropyltrimethylammonium chloride, and hydroxyethylguar hydroxypropyltrimonium chloride and the one or more polyquaterniums are preferably selected from polyquaternium-4, polyquaternium-7, polyquaternium-10, and combinations thereof; and
(f) optionally, about 0.05 to about 4 wt. %, preferably about 0.1 to about 3 wt. %, and more preferably about 0.5 to about 2 wt. % of one or more pearlizing agents, wherein the one or more pearlizing agents are preferably selected from glycol distearate, PEG-150 distearate, sorbitan stearate, mica, titanium dioxide, alumina, or combinations thereof, more preferably wherein the pearlizing agent is glycol distearate, PEG-150 distearate, or a combination thereof;
(g) optionally, about 0.01 to about 3 wt. %, preferably about 0.05 to about 2 wt. %, and more preferably about 0.1 to about 1 wt. % of one or more thickening polymers, preferably one or more nonionic thickening polymers, wherein the one or more nonionic thickening polymers are preferably selected from polyacrylate polymers, polyacrylamide polymers, carbomer, polysaccharides or derivatives thereof (polysaccharide compounds), and gums;
(h) optionally, less than 10 wt. %, preferably less than 5 wt. %, more preferably less than 2 wt. %, and even more preferably less than 1 wt. % of one or more water soluble solvents;
(i) optionally, less than 2 wt. %, preferably less than 1 wt. %, and more preferably less than 0.5 wt. % of one or more silicones, wherein preferably, if the cleansing composition includes one or more silicones, the one or more silicones are selected from amino-silicones, preferably amodimethicone;
(j) optionally, about 0.001 to about 5 wt. %, preferably about 0.01 to about 3 wt. %, more preferably about 0.1 to about 2 wt. % of one or more direct dyes;
(k) optionally, about 0.1 to about 10 wt. %, preferably about 0.5 to about 8 wt. %, and even more preferably about 1 to about 5 wt. % of one or more miscellaneous ingredients, wherein the one or more miscellaneous ingredients are preferably selected from pH adjusters, salts, fragrances, preservatives, antioxidants, skin active agents, anti-wrinkle agent, UV filters, de-pigmenting agents, chelating agents, compositions colorants, fillers, humectants, emollients, botanical extracts, amino acids, peptides, proteins, or a combination thereof;
   wherein the composition is free or essentially from sulfate-based anionic surfactants;
   wherein preferably, the composition has a pH of about 4 to less than 7, more preferably about 4.5 to about 6.5, and even more preferably about 5.5 to about 6.2;
   wherein optionally, the composition preferably comprises less than 2 wt. %, more preferably less than 1 wt. %, and even more preferably less than 0.5 wt. % of C₁-C₆ monoalcohols and all percentages by weight are based on the total weight of the cleansing composition.

In another preferred embodiments, the cleansing composition comprises, consists essentially of, or consists of:
(a) about 2 to about 20 wt. %, preferably about 3 to about 15 wt. %, more preferably about 4 to about 12 wt. % of a plurality of non-sulfate anionic surfactants, the plurality of non-sulfate anionic surfactants comprising, consisting essentially of, or consisting of:
   (i) about 1 to about 12 wt. %, preferably about 2 to about 10 wt. %, more preferably about 3 to about 8 wt. % of one or more isethionate surfactants selected from sodium cocoyl isethionate, sodium lauroyl isethionate, sodium myristoyl isethionate, sodium capryloyl isethionate, sodium stearoyl isethionate, sodium oleoyl isethionate, sodium palmitoyl isethionate, sodium lauroyl methyl isethionate, sodium cocoyl methyl isethionate, sodium decanoyl isethionate, sodium lauroyl sarcosinate, sodium cetyl isethionate, sodium isethionate, sodium cocoate isethionate, sodium hexanoyl isethionate, sodium tetradecanoyl isethionate, sodium nonanoyl isethionate, sodium oleoyl methyl isethionate, sodium isethionate laurate, sodium isethionate stearate, sodium dodecanoyl isethionate, sodium palmitoleoyl isethionate, sodium isethionate caprate, sodium isethionate linoleate, sodium lauryl isethionate, sodium caproyl isethionate, and sodium isethionate behenate, and even more preferably, the one or more isethionate surfactants comprises or consists of sodium cocoyl isethionate;
   (ii) about 0.5 to about 12 wt. %, preferably about 1 to about 10 wt. %, more preferably about 1 to about 5 wt. % of one or more sarcosinate surfactants selected from sodium lauroyl sarcosinate, sodium cocoyl sarcosinate, sodium myristoyl sarcosinate, sodium palmitoyl sarcosinate, sodium stearoyl sarcosinate, sodium oleoyl sarcosinate, sodium linoleoyl sarcosinate, sodium linolenoyl sarcosinate, sodium capryloyl sarcosinate, sodium caproyl sarcosinate, sodium lauric acid sarcosinate, sodium lauroyl methyl sarcosinate, sodium cocoyl methyl sarcosinate, sodium myristoyl methyl sarcosinate, sodium palmitoyl methyl sarcosinate, sodium stearoyl methyl sarcosinate, sodium oleoyl methyl sarcosinate, sodium linoleoyl methyl sarcosinate, sodium linolenoyl methyl sarcosinate, sodium capryloyl methyl sarcosinate, sodium caproyl methyl sarcosinate, sodium lauryl sarcosinate, sodium cocoyl sarcosinate, sodium myristyl sarcosinate, sodium palmityl sarcosinate, sodium stearyl sarcosinate, sodium oleyl sarcosinate, sodium linoleyl sarcosinate, sodium linolenyl sarcosinate, sodium caprylyl sarcosinate, and sodium caprolyl sarcosinate, wherein even more preferably, the one or more sarcosinate surfactants comprises or consists of sodium lauroyl sarcosinate;
   (iii) about 0.1 to about 5 wt. %, preferably about 0.2 to about 4 wt. %, more preferably about 0.5 to about 3 wt. % of one or more alkoxylated monoacid surfactants selected from trideceth-7 carboxylic acid, sodium laureth-13 carboxylate, sodium laureth-4 carboxylate, laureth-11 carboxylic acid, laureth-5 carboxylic acid, sodium laureth-5 carboxylate, ammonium laureth-6 carboxylate, ammonium laureth-8 carboxylate, capryleth-4 carboxylic acid, capryleth-6 carboxylic acid, capryleth-9 carboxylic acid, ceteareth-25 carboxylic acid, cetyl c12-15 pareth-8 carboxylate, cetyl C12-15 pareth-9 carboxylate, cetyl ppg-2 isodeceth-7 carboxylate, coceth-7 carboxylic acid, C9-11 pareth-6 carboxylic acid, C9-11 pareth-8 carboxylic acid, C11-15 pareth-7 carboxylic acid, C12-13 pareth-5 carboxylic acid, C12-13 pareth-7 carboxylic acid, C12-13 pareth-8 carboxylic acid, C12-13 pareth-12 carboxylic acid, C12-15 pareth-7 carboxylic acid, C12-15 pareth-8 carboxylic acid, C12-15 pareth-12 carboxylic acid, C14-15 pareth-8 carboxylic acid, deceth-7 carboxylic acid, ethylhexeth-3 carboxylic acid, hexeth-4 carboxylic acid, isopropyl C12-15-pareth-9 carboxylate, isopropyl ppg-2 isodeceth-7 carboxylate, isosteareth-6 carboxylic acid, isosteareth-11 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, magnesium laureth-11 carboxylate, mea-laureth-6 carboxylate, mea ppg-6 laureth-7 carboxylate, mea-ppg-8-stearath-7 carboxylate, myreth-3 carboxylic acid, myreth-5 carboxylic acid, oleth-3 carboxylic acid, oleth-6 carboxylic acid, oleth-10 carboxylic acid, peg-2 stearamides carboxylic acid, peg-9 stearamide carboxylic acid, potassium laureth-3 carboxylate, potassium laureth-4 carboxylate, potassium laureth-5 carboxylate, potassium laureth-6 carboxylate, potassium laureth-10 carboxylate, potassium trideceth-3 carboxylate, potassium trideceth-4 carboxylate, potassium trideceth-7 carboxylate, potassium trideceth-15 carboxylate, potassium trideceth-19 carboxylate, ppg-3-deceth-2 carboxylic acid, propyl c12-15 pareth-8 carboxylate, sodium capryleth-2 carboxylate, sodium capryleth-9 carboxylate, sodium ceteareth-13 carboxylate, sodium ceteth-13 carboxylate, sodium C9-11 pareth-6 carboxylate, sodium C11-15 pareth-7 carboxylate, sodium C12-13 pareth-5 carboxylate, sodium C12-13 pareth-8 carboxylate, sodium C12-13 pareth-12 carboxylate, sodium C12-15 pareth-6 carboxylate, sodium C12-15 pareth-7 carboxylate, sodium C12-15 pareth-8 carboxylate, sodium C12-15 pareth-12 carboxylate, sodium C14-15 pareth-8 carboxylate, sodium C12-14 sec-pareth-8 carboxylate, sodium deceth-2 carboxylate, sodium hexeth-4 carboxylate, sodium isosteareth-6 carboxylate, sodium isosteareth-11 carboxylate, sodium laureth-3 carboxylate, sodium laureth-4 carboxylate, sodium laureth-5 carboxylate, sodium laureth-6 carboxylate, sodium laureth-8 carboxylate, sodium laureth-11 carboxylate, sodium laureth-12 carboxylate, sodium laureth-13 carboxylate, sodium laureth-14 carboxylate, sodium laureth-16 carboxylate, sodium laureth-17 carboxylate, sodium lauryl glucose carboxylate, sodium lauryl glycol carboxylate, sodium peg-6 cocamide carboxylate, sodium peg-8 cocamide carboxylate, sodium peg-3 lauramide carboxylate, sodium peg-4 lauramide carboxylate, sodium peg-7 olive oil carboxylate, sodium peg-8 palm glycerides carboxylate, sodium trideceth-3 carboxylate, sodium trideceth-4 carboxylate, sodium trideceth-6 carboxylate, sodium trideceth-7 carboxylate, sodium trideceth-8 carboxylate, sodium trideceth-12 carboxylate, sodium trideceth-15 carboxylate, sodium trideceth-19 carboxylate, sodium undeceth-5 carboxylate, trideceth-3 carboxylic acid, trideceth-4 carboxylic acid, trideceth-7 carboxylic acid, trideceth-8 carboxylic acid, trideceth-15 carboxylic acid, trideceth-19 carboxylic acid and undeceth-5 carboxylic acid, wherein preferably, the one or more alkoxylated monoacid surfactants is laureth-5 carboxylic acid;
      wherein (a) (ii) and (a) (iii) are preferably in a combined amounts of about 0.2 to less than 5 wt. %, more preferably about 0.5 to about 4 wt. %, and even more preferably about 0.5 to about 3 wt. %; and
   (iv) optionally, about 0.01 to about 10 wt. %, about 0.05 to about 6 wt. %, or about 0.1 to about 5 wt. % of one or more additional non-sulfate anionic surfactants;
(b) about 1 to about 20 wt. %, preferably about 2 to about 15 wt. %, more preferably about 5 to about 12 wt. %, and even more preferably about 6 to about 10 wt. % of one or more betaine surfactants selected from coco betaine, cocamidopropyl betaine, lauryl betaine, laurylhydroxy sulfobetaine, lauryldimethyl betaine, behenyl betaine, capryl/capramidopropyl betaine, stearyl betaine, lauramidopropyl betaine, myristamidopropyl betaine, palmitamidopropyl betaine, stearamidopropyl betaine, oleamidopropyl betaine, capramidopropyl betaine, caprylamidopropyl betaine, behenamidopropyl betaine, lauramidopropyl betaine, cocamidopropyl hydroxysultaine, and cocamidopropyl dimethylamine betaine;
   wherein (a) and (b) are in a weight ratio of about 3:1 to about 1:3, preferably about 2:1 to about 1:2, and more preferably about 1.3:1 to greater than 1:1.3;
(c) less than 5 wt. %, more preferably less than 3 wt. %, and more preferably less than 2 wt. % of one or more nonionic surfactants, wherein preferably, the cleansing compositions is free or essentially free from polyglucoside surfactants:
(d) about 60 to about 90 wt. %, preferably about 65 to about 88 wt. %, more preferably about 70 to about 85 wt. % of water; and
(e) optionally, about 0.1 to about 5 wt. %, preferably about 0.1 to about 4 wt. %, more preferably about 0.2 to about 2 wt. % of one or more, preferably two or more, cationic conditioning polymers, wherein the one or more (or preferably two or more) cationic guar compounds are selected from cationic guar compounds, polyquaterniums, or combinations thereof and the one or more cationic guar compounds are selected from guar hydroxypropyltrimonium chloride, guar hydroxypropyltrimethylammonium chloride (GPTC), hydroxypropyl guar hydroxypropyltrimonium chloride, guar hydroxypropyltrimethylammonium chloride, and hydroxyethylguar hydroxypropyltrimonium chloride and the one or more polyquaterniums are preferably selected from polyquaternium-4, polyquaternium-7, polyquaternium-10, and combinations thereof;
(f) optionally, about 0.05 to about 4 wt. %, preferably about 0.1 to about 3 wt. %, and more preferably about 0.5 to about 2 wt. % of one or more pearlizing agents, wherein the one or more pearlizing agents are preferably selected from glycol distearate, PEG-150 distearate, sorbitan stearate, mica, titanium dioxide, alumina, or combinations thereof, more preferably wherein the pearlizing agent is glycol distearate, PEG-150 distearate, or a combination thereof;
(g) optionally, about 0.01 to about 3 wt. %, preferably about 0.05 to about 2 wt. %, and more preferably about 0.1 to about 1 wt. % of one or more thickening polymers, preferably one or more nonionic thickening polymers, wherein the one or more nonionic thickening polymers are preferably selected from polyacrylate polymers, polyacrylamide polymers, carbomer, polysaccharides or derivatives thereof (polysaccharide compounds), and gums;
(h) optionally, less than 10 wt. %, preferably less than 5 wt. %, more preferably less than 2 wt. %, and even more preferably less than 1 wt. % of one or more water soluble solvents;
(i) optionally, less than 2 wt. %, preferably less than 1 wt. %, and more preferably less than 0.5 wt. % of one or more silicones, wherein preferably, if the cleansing composition includes one or more silicones, the one or more silicones are selected from amino-silicones, preferably amodimethicone;
(j) optionally, about 0.001 to about 5 wt. %, preferably about 0.01 to about 3 wt. %, more preferably about 0.1 to about 2 wt. % of one or more direct dyes;
(k) optionally, about 0.1 to about 10 wt. %, preferably about 0.5 to about 8 wt. %, and even more preferably about 1 to about 5 wt. % of one or more miscellaneous ingredients, wherein the one or more miscellaneous ingredients are preferably selected from pH adjusters, salts, fragrances, preservatives, antioxidants, skin active agents, anti-wrinkle agent, UV filters, de-pigmenting agents, chelating agents, compositions colorants, fillers, humectants, emollients, botanical extracts, amino acids, peptides, proteins, or a combination thereof;
   wherein the composition is free or essentially from sulfate-based anionic surfactants;
   wherein preferably, the composition has a pH of about 4 to less than 7, more preferably about 4.5 to about 6.5, and even more preferably about 5.5 to about 6.2;
   wherein optionally, the composition preferably comprises less than 2 wt. %, more preferably less than 1 wt. %, and even more preferably less than 0.5 wt. % of C1-C6 monoalcohols and all percentages by weight are based on the total weight of the cleansing composition.

### EXAMPLES

Various changes can be made in the above-compositions and methods without departing from the scope of the invention. Accordingly, it is intended that the entire disclosure in the above description and in the examples given below, is illustrative and not limiting.

### Example 1

### Inventive Shampoo Compositions

TABLE 1

| | | A | B | C | D | E | F |
|---|---|---|---|---|---|---|---|
| (a)(i) | SODIUM COCOYL ISETHIONATE | 5 | 7 | 9 | 5 | 4 | 4 |
| (a)(ii) | SODIUM LAUROYL SARCOSINATE | 1.1 | 1.5 | 1 | 0.8 | 3 | 5 |
| (a)(iii) | LAURETH-5 CARBOXYLIC ACID | 0.9 | 0.5 | 1 | 4 | 0.5 | 3 |
| Total Anionic Surfactant | | 7 | 9 | 11 | 9.8 | 7.5 | 12 |
| (b) | COCAMIDOPROPYL BETAINE, | 8.8 | 8 | 5.5 | 6.5 | 9.1 | 4 |
| | COCO-BETAINE, AND/OR LAURYL | | | | | | |
| | BETAINE | | | | | | |
| Total Betaine Surfactant | | 8.8 | 8 | 5.5 | 6.5 | 9.1 | 4 |
| (c) | LAURETH-4, | 0.2 | 0.5 | 0.3 | 0.4 | 1.7 | 1.7 |
| | PPG-5-CETETH-20, | | | | | | |
| | COCAMIDE MEA, AND/OR | | | | | | |
| | PEG-55 PROPYLENE GLYCOL OLEATE | | | | | | |
| Total Nonionic Surfactant | | 0.2 | 0.5 | 0.3 | 0.4 | 1.7 | 1.7 |
| TOTAL SURFACTANT | | 16 | 17.5 | 16.8 | 16.7 | 18.3 | 17.7 |
| (d) | WATER | QS100 | | | | | |
| (e) | HYDROXYPROPYL GUAR | | | | 0.2 | 0.2 | 0.3 |
| | HYDROXYPROPYLTRIMONIUM CHLORIDE | | | | | | |
| | POLYQUATERNIUM-7 | 0.4 | 0.4 | 0.4 | | | |
| | POLYQUATERNIUM-10 | 0.1 | 0.1 | 0.1 | 0.2 | 0.2 | 0.2 |
| (f) | GLYCOL DISTEARATE AND/OR | 0.6 | 0.6 | 0.6 | 0.6 | 2 | 2 |
| | PEG-150 DISTEARATE | | | | | | |
| (g) | CETYL HYDROXYETHYLCELLULOSE | | | | | | 0.1 |
| (h) | PROPYLENE GLYCOL | 0.2 | 0.5 | 0.3 | 0.1 | | |
| (i) | AMODIMETHICONE | 0.4 | 0.4 | 0.4 | | | |
| (j) | SALICYLIC ACID | 0.2 | 0.2 | 0.2 | 0.2 | 0.2 | 0.2 |
| (k) | MISCELLANEOUS | ≤5 | ≤5 | ≤5 | ≤5 | ≤5 | ≤5 |

| | |
|---|---|
| pH | ~6 |
| Viscosity at 25° C. | ~3,000-15,000 cPs |

TABLE 2

| | | G | H | I | J | K | L |
|---|---|---|---|---|---|---|---|
| (a)(i) | SODIUM COCOYL ISETHIONATE | 5 | 4 | 8 | 5 | 5 | 5 |
| (a)(ii) | SODIUM LAUROYL SARCOSINATE | 1.1 | 1.1 | 1.1 | 5 | 1.1 | 1.1 |
| (a)(iii) | LAURETH-5 CARBOXYLIC ACID | 1.4 | 0.9 | 0.9 | 0.5 | 0.9 | 2.0 |
| Total Anionic Surfactant | | 7.5 | 6 | 10 | 10.5 | 7 | 8.1 |
| (b) | COCAMIDOPROPYL BETAINE | 8 | 6 | 9 | 5 | 9.1 | 9.1 |
| | AND COCO-BETAINE | | | | | | |
| Total Betaine Surfactant | | 8 | 6 | 9 | 5 | 9.1 | 9.1 |
| (c) | COCAMIDE MEA AND LAURETH-4 | 1.6 | 1.3 | 1 | 1 | 1.6 | 1.2 |
| Total Nonionic Surfactant | | 1.6 | 1.3 | 1 | 1 | 1.6 | 1.1 |
| TOTAL SURFACTANT | | 17.1 | 13.3 | 20 | 16.5 | 17.7 | 18.3 |
| | POLYQUATERNIUM-10 | 0.2 | 0.2 | 0.2 | 0.2 | 0.2 | 0.2 |
| (d) | WATER | QS100 | | | | | |
| (e) | HYDROXYPROPYL GUAR | 0.2 | 0.2 | 0.2 | 0.2 | 0.2 | 0.2 |
| | HYDROXYPROPYLTRIMONIUM | | | | | | |
| | CHLORIDE | | | | | | |
| (f) | PEG-150 DISTEARATE AND | 2 | 2 | 2 | 2 | 2 | 2 |
| | GLYCOL DISTEARATE | | | | | | |
| (g) | CARBOMER | | | | 0.1 | | |
| (h) | GLYCERIN | | | | | 2 | |
| (i) | OPTIONAL, SILICONE | | | | | | |
| (j) | MISCELLANEOUS | ≤5 | ≤5 | ≤5 | ≤5 | ≤5 | ≤5 |

| | |
|---|---|
| pH | ~6 |
| Viscosity at 25° C. | ~3,000-15,000 cPs |

### Example 2

Inventive Shampoo Compositions with Direct Dyes

TABLE 3

| | | M | N | O | P |
|---|---|---|---|---|---|
| (a)(i) | SODIUM COCOYL | 5 | 10 | 4 | 8 |
| | ISETHIONATE | | | | |
| (a)(ii) | SODIUM LAUROYL | 1.1 | 0.8 | 0.5 | 1 |
| | SARCOSINATE | | | | |
| (a)(iii) | LAURETH-5 CARBOXYLIC | 0.9 | 0.5 | 1.5 | 1 |
| | ACID | | | | |
| Total Anionic Surfactant | | 7 | 11.3 | 6 | 10 |
| (b) | COCAMIDOPROPYL | 9 | 5.3 | 10.1 | 4.1 |
| | BETAINE & COCO-BETAINE | | | | |
| Total Betaine Surfactant | | 9 | 5.3 | 10.1 | 4.1 |
| (c) | PPG-5-CETETH-20 & | 0.5 | 0.5 | 0.2 | 0.2 |
| | PEG-55 PROPYLENE | | | | |
| | GLYCOL OLEATE | | | | |
| Total Nonionic Surfactant | | 0.5 | 0.5 | 0.2 | 0.2 |
| TOTAL SURFACTANT | | 16.5 | 17.1 | 16.3 | 14.3 |
| (d) | WATER | QS100 | | | |
| (e) | POLYQUATERNIUM-7 | 0.4 | 0.4 | 0.4 | 0.4 |
| | POLYQUATERNIUM-10 | 0.1 | 0.1 | 0.1 | 0.1 |
| (f) | GLYCOL DISTEARATE | 1.6 | 1.6 | 0.6 | 0.6 |
| (g) | CARBOMER | 0.2 | 0.2 | | |
| (h) | PROPYLENE GLYCOL | 0.3 | 0.3 | 0.2 | 0.2 |
| (i) | AMODIMETHICONE | 0.4 | 0.4 | 0.4 | 0.4 |
| (j) | HC VIOLET NO. 2 | | | | 0.2 |
| | EXT. VIOLET 2 | 0.5 | 0.5 | | |
| | BASIC RED 51 | | | 0.6 | 0.0 |
| | BASIC YELLOW 87 | | | 0.7 | 0.2 |
| (k) | MISCELLANEOUS | ≤5 | ≤5 | ≤5 | ≤5 |

| | |
|---|---|
| pH | ~6 |
| Viscosity at 25° C. | ~3,000-15,000 cPs |

### Example 3

### Foam Testing

TABLE 4

| | | W | C-W |
|---|---|---|---|
| (a)(i) | SODIUM COCOYL ISETHIONATE | 5 | 5 |
| (a)(ii) | SODIUM LAUROYL SARCOSINATE | 1.1 | 1.1 |
| (a)(iii) | LAURETH-5 CARBOXYLIC ACID | 3 | |
| Total Anionic Surfactant | | 9.1 | 6.1 |
| (b) | COCAMIDOPROPYL BETAINE AND | 9.1 | 9.1 |
| | COCO-BETAINE | | |
| Total Betaine Surfactant | | 9.1 | 9.1 |
| (c) | LAURETH-4 AND COCAMIDE MEA | 1.6 | 1.6 |
| Total Nonionic Surfactant | | 1.6 | 1.6 |
| TOTAL SURFACTANT | | 19.8 | 16.8 |
| (d) | WATER | QS100 | |
| (e) | HYDROXYPROPYL GUAR | 0.2 | 0.2 |
| | HYDROXYPROPYLTRIMONIUM | | |
| | CHLORIDE | | |
| (f) | PEG-150 DISTEARATE AND | 2 | 2 |
| | GLYCOL DISTEARATE | | |
| (g) | SALICYLIC ACID | 0.2 | 0.2 |
| (h) | MISCELLANEOUS | ≤5 | ≤5 |

| | |
|---|---|
| pH | ~6 |
| Viscosity at 25° C. | ~3,000-15,000 cPs |

Inventive composition W was evaluated against comparative composition C-W, which is identical to inventive composition W but does not include the alkoxylated monoacid cosurfactant (laureth-5 carboxylic acid). Both compositions were tested on the hair of a mannequin head. To ensure uniformity, the hair was first washed with a standard shampoo. After rinsing the shampoo, inventive composition W and comparative composition C-W were applied in equal amounts to opposite halves of the damp hair. The products were massaged into the hair for 30 seconds. The amount of foam produced at 2 seconds (termed “flash foam”) and at 30 seconds (referred to as “foam quantity”) was visually assessed. “Flash foam” denotes the rapid and abundant lather that forms when the shampoo is agitated or mixed with water. This quick foaming action is desirable, as it enhances the user experience by conveying the impression of a rich and effective product. “Foam quantity” indicates the total volume of foam generated, representing the lather. Inventive composition W exhibited superior flash foam and greater foam quantity compared to comparative composition C-W, which is unexpected, given that alkoxylated monoacid cosurfactants are not typically recognized for providing significant foaming properties in formulations.

The foregoing disclosure illustrates and describes embodiments of the invention. The disclosure shows and describes only the preferred embodiments, but it is understood that the invention is useable in various other combinations, modifications, and environments and is capable of changes or modifications within the scope of the inventive concepts expressed herein, commensurate with the above teachings and/or the skill or knowledge of the relevant art. The embodiments described above are further intended to explain best modes known by applicant and to enable others skilled in the art to utilize the disclosure. This description is not intended to limit the invention.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” or “a combination thereof” also relates to “mixtures thereof” or “combinations thereof.” Throughout the disclosure, the term “a combination thereof” may be used following a list of elements as shown in the following example where letters A-F represent the elements: “one or more elements selected from A, B, C, D, E, F, or combination thereof.” The term, “combination thereof” does not require that the combination include all of A, B, C, D, E, and F (although all of A, B, C, D, E, and F may be included). Rather, it indicates that a combination of any two or more of A, B, C, D, E, and F can be included. In other words, it is equivalent to the phrase “one or more elements selected from A, B, C, D, E, F, and a combination of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a combination thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The surfactants referred to throughout the disclosure may include those having a counterion such as an alkali metal, alkaline earth metal, or ammonium counterion. This list of counterions, however, is nonlimiting. Regardless of whether the possibility of a salt for a particular component is mentioned, both the salt and the non-salt form of the component are intended, unless stated otherwise. In other words, all listed surfactants include a counterion, where applicable. Although the specific counterion may not be explicitly mentioned for each surfactant, it is understood that each surfactant is typically associated with a counterion such as sodium, potassium, calcium, or ammonium, depending on the chemical formulation and intended application. This inclusion of a counterion ensures the proper ionic balance and functionality of the surfactant. For example, if reference is made to a surfactant term such as “lauryl sulfoacetate,” it is understood that this term encompasses surfactants with various counterions, such as “sodium lauryl sulfoacetate.” Conversely, if the term “sodium lauryl sulfoacetate” is used, it includes the broader category of “lauryl sulfoacetate,” for example, with sodium as a counterion. This ensures clarity in understanding that the counterion may be implied based on the context or formulation of the surfactant.

The expression “one or more” means “at least one” and thus includes individual components as well as mixtures/combinations.

The term “plurality” means “more than one” or “two or more.”

Other than in the operating examples, or where otherwise indicated, all numbers expressing quantities of ingredients and/or reaction conditions can be modified in all instances by the term “about,” meaning within +/−10% of the indicated number. For example, an amount of “about 1 wt.” can include an amount as low as 0.90 wt. % or as high as 1.1. Similarly, an amount of “about 50” can include an amount as low as 45 wt. % and as high as 55 wt. %.

Some of the various categories of ingredients identified for the cleansing composition may overlap. In such cases where overlap may exist and the cleansing composition includes two overlapping ingredients (or more than two overlapping ingredients), an overlapping ingredient does not represent more than one claimed component. As an example, certain fatty esters are known in the art as both a “surfactant” and a “fatty compound.” If a claimed composition references both a surfactant and a fatty compound, the fatty ester serves as only the surfactant or as only the fatty compound. The fatty ester cannot simultaneously serve as both the surfactant and the fatty compound.

All percentages, parts, and ratios herein are based upon the total weight of the compositions, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

All components positively set forth in the instant disclosure can be negatively excluded from the claims and from the compositions and methods described throughout the disclosure In other words, the compositions of the instant disclosure may be free or essentially free of any one or more of the components positively set forth in the instant disclosure for possible inclusion into the compositions.

The term “substantially free” or “essentially free” as used herein means the specific material may be present in small amounts that do not materially affect the basic and novel characteristics of the claimed invention. For instance, there may be less than 1% by weight of a specific material added to a composition, based on the total weight of the compositions (provided that an amount of 1% or less by weight does not materially affect the basic and novel characteristics of the claimed invention). Similarly, when a composition is essentially free from a particular element, the composition may include 1 wt. % or less, 0.5 wt. % or less, 0.1 wt. % or less, 0.05 wt. % or less, 0.01 wt. % or less, or none of the specified material. A composition that is essentially or substantially free from an element may include, for example, up to 1 wt. %, up to 0.9 wt. %, up to 0.8 wt. %, up to 0.7 wt. %, up to 0.6 wt. %, up to 0.5 wt. %, up to 0.4 wt. %, up to 0.3 wt. %, up to 0.2 wt. %, up to 0.1 wt. %, up to about 0.09 wt. %, up to 0.08 wt. %, up to 0.07 wt. %, up to 0.06 wt. %, or up to 0.05 wt. %, or from 0.05 to 1 wt. %, 0.05 to 0.9 wt. %, 0.05 to 0.8 wt. %, 0.05 to 0.7 wt. %, 0.05 to 0.6 wt. %, 0.05 to 0.5 wt. %, 0.05 to 0.4 wt. %, 0.05 to 0.3 wt. %, or 0.05 to 0.1 wt. %. As an example, if a composition is “substantially free” or “essentially free” from ethanol, the composition may optionally include ethanol in an amount up to 1 wt. %, based on the total weight of the composition, provided an amount of up to 1 wt. % does not materially affect the basic and novel characteristics of the composition.

The term “less than” a particular amount of a component encompasses the absence of the component. In other words, “less than” a particular amount of a component encompasses 0% of the component. For example, if a claim refers to an amount of less than 5 wt. % of component X, the claim does not require component X because 0 wt. % is encompasses by the phrase “less than 5 wt. %.””

The term “transparent,” with respect to a transparent composition, indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure. A human can typically see through a transparent composition, for example, and read the text on the other side of a clear glass or clear plastic bottle containing the composition.

The term “translucent,” with respect to a translucent composition, indicates that the composition has a transmittance of at least 50% at a wavelength of 600 nm, for example measured using a Lambda 40 UV-visible spectrometer. A human cannot likely see through a translucent composition, for example, and read the text on the other side of a clear glass or clear plastic bottle containing the composition. Rather, the text is usually blurred and difficult or not possible to read, yet movement and structure can normally be identified.

The term “opaque,” with respect to an opaque composition, indicates the composition is not transparent or translucent, i.e., has a transmittance of less than 50% at a wavelength of 600 nm, for example measured using a Lambda 40 UV-visible spectrometer.

All publications and patent applications cited throughout the disclosure are incorporated herein by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cleansing composition comprising:
(a) about 2 to about 25 wt. % of a plurality of non-sulfate anionic surfactants, the plurality of non-sulfate anionic surfactants comprising:
(i) about 1 to about 10 wt. % of one or more isethionate surfactants;
(ii) about 0.1 to about 10 wt. % of one or more amino acid surfactants; and
(iii) about 0.1 to about 5 wt. % of one or more alkoxylated monoacid cosurfactants;
(b) about 2 to about 10 wt. % of one or more amphoteric surfactants;
(c) optionally, one or more nonionic surfactants;
(d) about 60 to about 90 wt. % of water;
wherein the composition is substantially free from sulfate-based anionic surfactants, and all percentages by weight are based on a total weight of the cleansing composition.

2. The cleansing composition of claim 1, further comprising one or more cationic conditioning polymers.

3. The cleansing composition of claim 1, wherein the one or more isethionate surfactants are selected from sodium cocoyl isethionate, sodium lauroyl isethionate, sodium myristoyl isethionate, sodium capryloyl isethionate, sodium stearoyl isethionate, sodium oleoyl isethionate, sodium palmitoyl isethionate, sodium lauroyl methyl isethionate, sodium cocoyl methyl isethionate, sodium decanoyl isethionate, sodium cetyl isethionate, sodium isethionate, sodium cocoate isethionate, sodium hexanoyl isethionate, sodium tetradecanoyl isethionate, sodium nonanoyl isethionate, sodium oleoyl methyl isethionate, sodium isethionate laurate, sodium isethionate stearate, sodium dodecanoyl isethionate, sodium palmitoleoyl isethionate, sodium isethionate caprate, sodium isethionate linoleate, sodium lauryl isethionate, sodium caproyl isethionate, and sodium isethionate behenate.

4. The cleansing composition of claim 1, wherein the one or more amino acid surfactants are selected from sarcosinate surfactants, taurate surfactants, glycinate surfactants, and glutamate surfactants.

5. The cleansing composition of claim 4 comprising an sarcosinate surfactant selected from sodium lauroyl sarcosinate, sodium cocoyl sarcosinate, sodium myristoyl sarcosinate, sodium palmitoyl sarcosinate, sodium stearoyl sarcosinate, sodium oleoyl sarcosinate, sodium linoleoyl sarcosinate, sodium linolenoyl sarcosinate, sodium capryloyl sarcosinate, sodium caproyl sarcosinate, sodium lauric acid sarcosinate, sodium lauroyl methyl sarcosinate, sodium cocoyl methyl sarcosinate, sodium myristoyl methyl sarcosinate, sodium palmitoyl methyl sarcosinate, sodium stearoyl methyl sarcosinate, sodium oleoyl methyl sarcosinate, sodium linoleoyl methyl sarcosinate, sodium linolenoyl methyl sarcosinate, sodium capryloyl methyl sarcosinate, sodium caproyl methyl sarcosinate, sodium lauryl sarcosinate, sodium cocoyl sarcosinate, sodium myristyl sarcosinate, sodium palmityl sarcosinate, sodium stearyl sarcosinate, sodium oleyl sarcosinate, sodium linoleyl sarcosinate, sodium linolenyl sarcosinate, sodium caprylyl sarcosinate, and sodium caprolyl sarcosinate.

6. The cleansing composition of claim 1, wherein the one or more alkoxylated monoacid cosurfactants are selected from trideceth-7 carboxylic acid, sodium laureth-13 carboxylate, sodium laureth-4 carboxylate, laureth-11 carboxylic acid, laureth-5 carboxylic acid, sodium laureth-5 carboxylate, ammonium laureth-6 carboxylate, ammonium laureth-8 carboxylate, capryleth-4 carboxylic acid, capryleth-6 carboxylic acid, capryleth-9 carboxylic acid, ceteareth-25 carboxylic acid, cetyl c12-15 pareth-8 carboxylate, cetyl C12-15 pareth-9 carboxylate, cetyl ppg-2 isodeceth-7 carboxylate, coceth-7 carboxylic acid, C9-11 pareth-6 carboxylic acid, C9-11 pareth-8 carboxylic acid, C11-15 pareth-7 carboxylic acid, C12-13 pareth-5 carboxylic acid, C12-13 pareth-7 carboxylic acid, C12-13 pareth-8 carboxylic acid, C12-13 pareth-12 carboxylic acid, C12-15 pareth-7 carboxylic acid, C12-15 pareth-8 carboxylic acid, C12-15 pareth-12 carboxylic acid, C14-15 pareth-8 carboxylic acid, deceth-7 carboxylic acid, ethylhexeth-3 carboxylic acid, hexeth-4 carboxylic acid, isopropyl C12-15-pareth-9 carboxylate, isopropyl ppg-2 isodeceth-7 carboxylate, isosteareth-6 carboxylic acid, isosteareth-11 carboxylic acid, laureth-3 carboxylic acid, laureth-4 carboxylic acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, magnesium laureth-11 carboxylate, mea-laureth-6 carboxylate, mea ppg-6 laureth-7 carboxylate, mea-ppg-8-stearath-7 carboxylate, myreth-3 carboxylic acid, myreth-5 carboxylic acid, oleth-3 carboxylic acid, oleth-6 carboxylic acid, oleth-10 carboxylic acid, peg-2 stearamides carboxylic acid, peg-9 stearamide carboxylic acid, potassium laureth-3 carboxylate, potassium laureth-4 carboxylate, potassium laureth-5 carboxylate, potassium laureth-6 carboxylate, potassium laureth-10 carboxylate, potassium trideceth-3 carboxylate, potassium trideceth-4 carboxylate, potassium trideceth-7 carboxylate, potassium trideceth-15 carboxylate, potassium trideceth-19 carboxylate, ppg-3-deceth-2 carboxylic acid, propyl c12-15 pareth-8 carboxylate, sodium capryleth-2 carboxylate, sodium capryleth-9 carboxylate, sodium ceteareth-13 carboxylate, sodium ceteth-13 carboxylate, sodium C9-11 pareth-6 carboxylate, sodium C11-15 pareth-7 carboxylate, sodium C12-13 pareth-5 carboxylate, sodium C12-13 pareth-8 carboxylate, sodium C12-13 pareth-12 carboxylate, sodium C12-15 pareth-6 carboxylate, sodium C12-15 pareth-7 carboxylate, sodium C12-15 pareth-8 carboxylate, sodium C12-15 pareth-12 carboxylate, sodium C14-15 pareth-8 carboxylate, sodium C12-14 sec-pareth-8 carboxylate, sodium deceth-2 carboxylate, sodium hexeth-4 carboxylate, sodium isosteareth-6 carboxylate, sodium isosteareth-11 carboxylate, sodium laureth-3 carboxylate, sodium laureth-4 carboxylate, sodium laureth-5 carboxylate, sodium laureth-6 carboxylate, sodium laureth-8 carboxylate, sodium laureth-11 carboxylate, sodium laureth-12 carboxylate, sodium laureth-13 carboxylate, sodium laureth-14 carboxylate, sodium laureth-16 carboxylate, sodium laureth-17 carboxylate, sodium lauryl glucose carboxylate, sodium lauryl glycol carboxylate, sodium peg-6 cocamide carboxylate, sodium peg-8 cocamide carboxylate, sodium peg-3 lauramide carboxylate, sodium peg-4 lauramide carboxylate, sodium peg-7 olive oil carboxylate, sodium peg-8 palm glycerides carboxylate, sodium trideceth-3 carboxylate, sodium trideceth-4 carboxylate, sodium trideceth-6 carboxylate, sodium trideceth-7 carboxylate, sodium trideceth-8 carboxylate, sodium trideceth-12 carboxylate, sodium trideceth-15 carboxylate, sodium trideceth-19 carboxylate, sodium undeceth-5 carboxylate, trideceth-3 carboxylic acid, trideceth-4 carboxylic acid, trideceth-7 carboxylic acid, trideceth-8 carboxylic acid, trideceth-15 carboxylic acid, trideceth-19 carboxylic acid and undeceth-5 carboxylic acid.

7. The cleansing composition of claim 1, wherein the one or more amphoteric surfactants are selected from betaine surfactants, sultaine surfactants, amphopropionate surfactants, and amphoacetate surfactants.

8. The cleansing composition of claim 5 comprising one or more betaine surfactants selected from coco betaine, cocamidopropyl betaine, lauryl betaine, laurylhydroxy sulfobetaine, lauryldimethyl betaine, behenyl betaine, capryl/capramidopropyl betaine, stearyl betaine, lauramidopropyl betaine, myristamidopropyl betaine, palmitamidopropyl betaine, stearamidopropyl betaine, oleamidopropyl betaine, capramidopropyl betaine, caprylamidopropyl betaine, behenamidopropyl betaine, lauramidopropyl betaine, cocamidopropyl hydroxysultaine, and cocamidopropyl dimethylamine betaine.

9. The cleansing composition of claim 1 comprising less than 5 wt. % of the one or more nonionic surfactants.

10. The cleansing composition of claim 1 comprising the one or more nonionic surfactants, wherein at least one of the one or more nonionic surfactants is an alkoxylated nonionic surfactant.

11. The cleansing composition of claim 10, wherein the at least one alkoxylated nonionic surfactant is an ethoxylated fatty ester, an ethoxylated fatty alcohol, an ethoxylated sorbitan ester, a polyglycerolated fatty ester, a polyglycerolated fatty alcohol, a polyglycerolated sorbitan ester, an alkoxylated alkanolamide, a polyglycerolated alkanolamide, or combinations thereof.

12. The cleansing composition of claim 2, wherein the one or more cationic conditioning polymers are selected from cationic polysaccharides and polyquaterniums.

13. The cleansing composition of claim 1, wherein (b) is in an amount greater than a combined amount of (a) and (c).

14. The cleansing composition of claim 1, wherein the cleansing composition further comprises one or more pearlizing agents.

15. The cleansing composition of claim 1, wherein the cleansing composition has a pH of about 4 to less than 7.

16. The cleansing composition of claim 1, wherein the cleansing composition further comprises one or more direct dyes.

17. The cleansing composition of claim 16, wherein the one or more direct dyes are selected from nitro dyes, anionic dyes, azo dyes, cationic dyes, triarylmethane dyes, and xanthene dyes.

18. A cleansing composition comprising:
(a) about 5 to about 25 wt. % of a plurality of non-sulfate anionic surfactants, the plurality of non-sulfate anionic surfactants comprising:
(i) about 1 to about 10 wt. % of one or more isethionate surfactants;
(ii) about 0.1 to about 10 wt. % of one or more amino-acid surfactants; and
(iii) about 0.1 to about 5 wt. % of one or more alkoxylated monoacid cosurfactants; and
(iv) optionally, one or more additional non-sulfate anionic surfactants;
(b) about 2 to about 10 wt. % of one or more amphoteric surfactants selected from betaine surfactants, sultaine surfactants, amphopropionate surfactants, and amphoacetate surfactants;
(c) less than 5 wt. % of one or more nonionic surfactants;
(d) about 60 to about 90 wt. % of water;
(e) optionally, about 0.1 to about 3 wt. % of one or more cationic conditioning polymers;
(f) optionally, about 0.05 to about 4 wt. % of one or more pearlizing agents;
(g) optionally, about 0.1 to about 10 wt. % of one or more miscellaneous ingredients selected from pH adjusters, salts, fragrances, preservatives, antioxidants, skin active agents, anti-wrinkle agent, UV filters, de-pigmenting agents, chelating agents, compositions colorants, fillers, humectants, emollients, botanical extracts, amino acids, peptides, proteins, or a combination thereof;
wherein the composition is substantially free from sulfate-based anionic surfactants, and all percentages by weight are based on a total weight of the cleansing composition.

19. A method for cleansing hair comprising applying the cleansing composition of claim 1 to the hair, and rinsing the cleansing composition from the hair.
